(19) United States
(12) Patent Application Publication
(10) Pub. No.: US 2021/0100769 A1
(43) Pub. Date: Apr. 8, 2021

(54) PRESERVATIVE-FREE FORMULATIONS OF TETRACAINE HCL

(71) Applicant: Altaire Pharmaceuticals, Inc., Aquebogue, NY (US)

(72) Inventor: Assad Sawaya, Baiting Hollow, NY (US)

(21) Appl. No.: 16/657,242

(22) Filed: Oct. 18, 2019

Related U.S. Application Data
(60) Provisional application No. 62/911,037, filed on Oct. 4, 2019.

(51) Int. Cl. A61K 31/245; A61K 9/00; A61K 47/02; A61K 47/18

(52) U.S. Cl. CPC A61K 31/245; A61K 47/183; A61K 47/02; A61K 9/0048

(57) ABSTRACT

Provided are liquid ophthalmic compositions containing tetracaine or a pharmaceutically acceptable salt thereof that do not contain a preservative. Also provided are methods of producing surface anesthesia of the eye.

### RELATED APPLICATIONS

This application claims the benefit of priority of U.S. Provisional Application No. 62/911,037, filed Oct. 4, 2019, which is incorporated herein by reference in its entirety.

### TECHNICAL FIELD

The present disclosure relates to preservative-free liquid ophthalmic compositions that contain tetracaine or a pharmaceutically acceptable salt thereof, and to the use of the compositions in producing surface anesthesia of the eye.

### BACKGROUND

Ophthalmic compositions containing drugs, such as anesthetics, are typically delivered to the eye through one of four modes of administration: systemic, topical, intravitreal, and periocular. Topical administration is generally considered the preferred route for the administration of ocular drugs due to its convenience and affordability. Drugs applied in this manner can be in multiple forms, including solutions, ointments, and suspensions. Liquid formulations are most commonly used for the eye, in part because they are the least expensive and interfere the least with vision. Some commonly used ocular medications are topical anesthetics, mydriatics and cycloplegics, medications used to treat glaucoma, anti-infectives, corticosteroids, and non-steroidal anti-inflammatory drugs.

The compound 2-(dimethylamino)ethyl 4-(butylamino)benzoate (tetracaine) has the following structure:

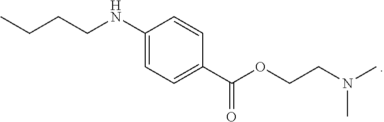 [SMILES: CCCCNc1ccc(C(=O)OCCN(C)C)cc1]

Tetracaine is a local anesthetic that can be used to numb the eye before and prevent pain during a number of surgeries, tests, or procedures associated with the eye. Tetracaine acts by decreasing the permeability of the neuronal membrane, thereby decreasing the flux of sodium, potassium and other ions associated with propagation of the nerve impulse. Compositions containing tetracaine, particularly tetracaine hydrochloride, are known. These compositions all contain preservatives, which are required for the composition to pass the United States Pharmacopeia (USP) microbial guidelines. The Food and Drug Administration (FDA) typically requires that multiple-dose containers of a drug product include a preservative to kill bacteria and fungi that may contaminate the fluid in the bottle; as a result, most medications have contained preservatives specifically designed to keep the medications sterile during patient use and the life cycle of the bottle.

Unfortunately, while these preservatives are effective against pathogens, they often are associated with deleterious effects on the eye because the chemicals that stop bacterial growth also can harm corneal and conjunctival epithelial cells. Long-term use of medications that contain these preservatives can lead problems such as dry-eye syndrome, foreign body sensation, and deteriorating vision, while short-term effects include irritation and possible allergic reaction.

Thus, there remains a need for an effective and safe topical ophthalmic pharmaceutical composition containing tetracaine or a pharmaceutically acceptable salt thereof with increased stability that avoids side effects. There is also a need for a method of producing surface anesthesia of the eye that avoids the irritation and side effects associated with the administration of a topical anesthetic that contains preservatives. In particular, there is a need for an ophthalmic composition that is free from preservatives to be provided in a multiple-use container and provide efficient dosing of the solution to the patient, without side effects.

### SUMMARY

Provided in this disclosure is a liquid ophthalmic composition the contains tetracaine or a pharmaceutically acceptable salt thereof at a concentration of about 0.2% to about 1.0% (w/v) disposed within a sterilized container suitable for a sterile ophthalmic product, wherein the composition is formulated for multiple-dose administration and the composition does not contain a preservative (i.e., is preservative-free). In some embodiments, the composition is for ophthalmic administration.

In some embodiments, the composition contains about 0.4% to about 0.6% (w/v) tetracaine or a pharmaceutically acceptable salt thereof. In some embodiments, the composition contains about 0.5% (w/v) tetracaine or a pharmaceutically acceptable salt thereof. In some embodiments, the tetracaine is tetracaine hydrochloride.

In some embodiments, the composition does not contain a preservative selected from the group consisting of chlorobutanol, benzalkonium chloride, PHMB, methylparaben, a stabilized peroxy complex, and any other pharmaceutically acceptable preservative for a sterile ophthalmic product.

In some embodiments, the composition contains a tonicity agent at a concentration of about 0.05% to about 0.5% (w/v). In some embodiments, the tonicity agent is at a concentration of about 0.108% (w/v). In some embodiments, the tonicity agent is potassium chloride.

In some embodiments, the composition contains a buffering agent at a concentration of about 0.1% to about 2% (w/v). In some embodiments, the buffering agent is at a concentration of about 1.24% (w/v). In some embodiments, the buffering agent is boric acid.

In some embodiments, the composition contains a chelating agent at a concentration of about 0.02% to about 0.08% (w/v). In some embodiments, the chelating agent is at a concentration of about 0.05% (w/v). In some embodiments, the chelating agent is edetate disodium.

In some embodiments, the pH of the composition is about 3 to about 6.5. In some embodiments, the pH of the composition is about 3.7 to about 6. In some embodiments, the pH of the composition is about 3 to about 5.5. In some embodiments, the pH of the composition is about 5.5.

In some embodiments, the composition is formulated as a total volume of about 1 mL to about 30 mL. In some embodiments, the composition is formulated as a total volume of about 2 mL to about 15 mL. In some embodiments, the composition is formulated as a total volume of about 1 mL, about 2 mL, about 4 mL, about 10 mL, about 15 mL or about 30 mL.

In some embodiments, the composition has been sterilized.

In some embodiments, the composition exhibits antimicrobial preservative efficacy against bacterial and fungal organisms selected from among Staphylococcus aureus, Pseudomonas aeruginosa, Escherichia coli, Candida albicans, and Aspergillus brasiliensis. In some embodiments, the composition exhibits not less than about a 1.0 log reduction from the initial calculated count at 7 days, not less than about a 3.0 log reduction from the initial calculated count at 14 days, and no increase from the 14 days' count at 28 days for bacteria; and no increase from the initial calculated count at 7 days, 14 days and 28 days for yeast and mold. In some embodiments, the antimicrobial preservative efficacy of the composition remains effective throughout the entire shelf-life of the product. In some embodiments, the shelf-life is at least about 6 months, at least about 12 months, at least about 24 months, at least about 36 months, at least about 48 months, or at least about 60 months.

Also disclosed herein is a liquid ophthalmic composition consisting of about 0.2% to about 1.0% (w/v) tetracaine or a pharmaceutically acceptable salt thereof; about 0.05% to about 0.5% (w/v) of a tonicity agent; about 0.1% to about 2% (w/v) of a buffering agent; about 0.02% to about 0.08% (w/v) of a chelating agent; a pH adjuster; and water; wherein the composition is disposed within a container-closure system suitable for a sterile ophthalmic product and is formulated for multiple-dose administration. In some embodiments, the tetracaine is tetracaine hydrochloride.

In some embodiments, the liquid ophthalmic composition consists of about 0.4% to about 0.6% (w/v) tetracaine hydrochloride; about 0.05% to about 0.5% (w/v) potassium chloride; about 0.1% to about 2% (w/v) boric acid; about 0.02% to about 0.08% (w/v) edetate disodium; a pH adjuster; and water; wherein the composition is disposed within a container-closure system suitable for a sterile ophthalmic product and is formulated for multiple-dose administration.

In some embodiments, the liquid ophthalmic composition consists of 0.5% (w/v) tetracaine hydrochloride; 0.108% (w/v) potassium chloride; 1.24% (w/v) boric acid; 0.05% (w/v) edetate disodium; a pH adjuster; and water; wherein the composition is disposed within a container-closure system appropriate for a sterile ophthalmic product and is formulated for multiple-dose administration.

In some embodiments, the liquid ophthalmic composition is for ophthalmic administration.

In some embodiments, the liquid ophthalmic composition is formulated as a total volume of about 1 mL to about 30 mL. In some embodiments, the liquid ophthalmic composition is formulated as a total volume of about 2 mL to about 15 mL. In some embodiments, the liquid ophthalmic composition is formulated as a total volume of about 1 mL, about 2 mL, about 4 mL, about 10 mL, about 15 mL or about 30 mL.

In some embodiments, the liquid ophthalmic composition has been sterilized.

Also provided in this disclosure is a method of producing surface anesthesia of the eye in a subject in need thereof, the method including administering a liquid ophthalmic composition comprising tetracaine or a pharmaceutically acceptable salt thereof at a concentration of about 0.2% to about 1.0% (w/v), wherein the composition is disposed within a container-closure system appropriate for a sterile ophthalmic product and is formulated for multiple-dose administration; and the composition does not contain a preservative.

In some embodiments of the method, the composition does not contain the preservative chlorobutanol, benzalkonium chloride, PHMB, methylparaben, a stabilized peroxy complex, or any other pharmaceutically acceptable preservative for a sterile ophthalmic product.

In some embodiments of the method, the composition contains about 0.4% to about 0.6% (w/v) tetracaine or a pharmaceutically acceptable salt thereof In some embodiments of the method, the composition contains about 0.5% (w/v) tetracaine hydrochloride.

In some embodiments of the method, the composition is formulated as a total volume of about 1 mL to about 30 mL. In some embodiments of the method, the composition is formulated as a total volume of about 2 mL to about 15 mL. In some embodiments of the method, the composition is formulated as a total volume of about 1 mL, about 2mL, about 4 mL, about 10 mL, about 15 mL, or about 30 mL.

In some embodiments of the method, the composition has been sterilized.

In some embodiments of the method, the composition is administered to the eye as an eye drop. In some embodiments of the method, a dose of one or two drops is administered. In some embodiments of the method, the dose is administered 1 to 3 times, 1 to 7 times, 1 to 5 times, 1 to 3 times, 1 to 2 times, 2 to 5 times, 2 to 3 times, 3 to 10 times, 3 to 7 times, or 3 to 5 times. In some embodiments of the method, the dose is administered one time, two times, three times, four times, five times, six times, seven times, eight times, nine times, or ten times. In some embodiments of the method, the dose is administered two or more times and is administered once about every 1-30 minutes, about every 1-15 minutes, about every 1-10 minutes, about every 1-5 minutes, about every 5-30 minutes, about every 5-15 minutes, about every 5-10 minutes, or about every 10-15 minutes. In some embodiments of the method, the dose is administered once about every 1 minute, 2 minutes, 3 minutes, 4 minutes, 5 minutes, 6 minutes, 7 minutes, 8 minutes, 9 minutes, 10 minutes, 15 minutes, 20 minutes, 25 minutes, or 30 minutes.

In some embodiments of the method, the composition is administered to the subject prior to evaluation of the subject for a procedure in which a rapid and short acting topical ophthalmic anesthetic is indicated. In some embodiments of the method, one dose of one or two drops is administered.

In some embodiments of the method, the composition is administered to the subject prior to the subject undergoing a procedure in which a rapid and short acting topical ophthalmic anesthetic is indicated.

In some embodiments of the method, the procedure is selected from the group consisting of tonometry, gonioscopy, removal of corneal foreign bodies, conjunctival scraping for diagnostic purposes, suture removal from the cornea or conjunctiva, cataract extraction, and other short corneal and conjunctival procedures. In some embodiments of the method, the procedure is tonometry or other short corneal and conjunctival procedures and one dose of one or two drops is administered to the subject prior to evaluation of the subject. In some embodiments of the method, the procedure is removal of corneal foreign bodies or suture removal from the cornea or conjunctiva and the dose is administered to the subject once about every 5-10 minutes for one to three times. In some embodiments of the method, the procedure is cataract extraction and the dose is administered to the subject once about every 5-10 minutes for three to five times.

Also provided in the present disclosure is a method of producing surface anesthesia of the eye in a subject in need thereof, the method comprising administering a liquid ophthalmic composition consisting of about 0.2% to about 1.0% (w/v) tetracaine or a pharmaceutically acceptable salt thereof; about 0.05% to about 0.5% (w/v) of a tonicity agent; about 0.1% to about 2% (w/v) of a buffering agent; about 0.02% to about 0.08% (w/v) of a chelating agent; a pH adjuster; and water; wherein the composition is disposed within a container-closure system appropriate for a sterile ophthalmic product and is formulated for multiple-dose administration. In some embodiments, the tetracaine is tetracaine hydrochloride.

In some embodiments of the method, the liquid ophthalmic composition consists of about 0.4% to about 0.6% (w/v) tetracaine hydrochloride; about 0.05% to about 0.5% (w/v) potassium chloride; about 0.1% to about 2% (w/v) boric acid; about 0.02% to about 0.08% (w/v) edetate disodium; a pH adjuster; and water.

In some embodiments of the method, the liquid ophthalmic composition consists of 0.5% (w/v) tetracaine hydrochloride; 0.108% (w/v) potassium chloride; 1.24% (w/v) boric acid; 0.05% (w/v) edetate disodium; a pH adjuster; and water.

In some embodiments of the method, the liquid ophthalmic composition is formulated as a total volume of about 1 mL to about 30 mL. In some embodiments of the method, the liquid ophthalmic composition is formulated as a total volume of about 2 mL to about 15 mL. In some embodiments of the method, the liquid ophthalmic composition is formulated as a total volume of about 1 mL, about 2 mL, about 4 mL, about 10 mL, about 15 mL, or about 30 mL.

In some embodiments of the method, the liquid ophthalmic composition has been sterilized.

In some embodiments of the method, the liquid ophthalmic composition is administered to the eye as an eye drop. In some embodiments of the method, a dose of one or two drops is administered. In some embodiments of the method, the dose is administered 1 to 3 times, 1 to 7 times, 1 to 5 times, 1 to 3 times, 1 to 2 times, 2 to 5 times, 2 to 3 times, 3 to 10 times, 3 to 7 times, or 3 to 5 times. In some embodiments of the method, the dose is administered one time, two times, three times, four times, five times, six times, seven times, eight times, nine times, or ten times. In some embodiments of the method, the dose is administered two or more times and is administered once about every 1-30 minutes, about every 1-15 minutes, about every 1-10 minutes, about every 1-5 minutes, about every 5-30 minutes, about every 5-15 minutes, about every 5-10 minutes, or about every 10-15 minutes. In some embodiments of the method, the dose is administered once about every 1 minute, 2 minutes, 3 minutes, 4 minutes, 5 minutes, 6 minutes, 7 minutes, 8 minutes, 9 minutes, 10 minutes, 15 minutes, 20 minutes, 25 minutes, or 30 minutes.

In some embodiments of the method, the composition is administered to the subject prior to evaluation of the subject for a procedure in which a rapid and short acting topical ophthalmic anesthetic is indicated. In some embodiments of the method, one dose of one or two drops is administered.

In some embodiments of the method, the composition is administered to the subject prior to the subject undergoing a procedure in which a rapid and short acting topical ophthalmic anesthetic is indicated.

In some embodiments of the method, the procedure is selected from the group consisting of tonometry, gonioscopy, removal of corneal foreign bodies, conjunctival scraping for diagnostic purposes, suture removal from the cornea or conjunctiva, and other short corneal and conjunctival procedures. In some embodiments of the method, the procedure is tonometry or other short corneal and conjunctival procedures and one dose of one or two drops is administered to the subject prior to evaluation of the subject. In some embodiments of the method, the procedure is removal of corneal foreign bodies or suture removal from the cornea or conjunctiva and the dose is administered to the subject once about every 5-10 minutes for one to three times. In some embodiments of the method, the procedure is cataract extraction and the dose is administered to the subject once about every 5-10 minutes for three to five times.

Other features and advantages of the processes, formulations, and uses provided herein will be apparent from the following detailed description and figures, and from the claims.

### DESCRIPTION OF DRAWINGS

FIG. 1 is a plot showing the S. aureus (ATCC 6538) plate count (cfu) as a function of kill time (minutes) with a preservative-free 0.5% tetracaine hydrochloride ophthalmic solution.

FIG. 2 is a plot showing the C. albicans (ATCC 10231) plate count (cfu) as a function of kill time (minutes) with a preservative-free 0.5% tetracaine hydrochloride ophthalmic solution.

FIG. 3 is a plot showing the P. aeruginosa (ATCC 9027) plate count (cfu) as a function of kill time (minutes) with a preservative-free 0.5% tetracaine hydrochloride ophthalmic solution.

FIG. 4 is a plot showing the A. brasiliensis (ATCC 16404) plate count (cfu) as a function of kill time (minutes) with a preservative-free 0.5% tetracaine hydrochloride ophthalmic solution.

FIG. 5 is a plot showing the E. coli (ATCC 8739) plate count (cfu) as a function of kill time (minutes) with a preservative-free 0.5% tetracaine hydrochloride ophthalmic solution.

### DETAILED DESCRIPTION

The eye is the most easily accessible site for topical administration of medications useful for producing surface anesthesia of the eye and for treatment of ophthalmic diseases and conditions. Ophthalmic preparations are typically sterile products that are essentially free from foreign particles, are suitably compounded, and are packaged for instillation into the eye. Ophthalmic preparations, such as aqueous ophthalmic formulations, are required to be sterile; thus, antimicrobial preservatives are typically added. For ophthalmic preparations formulated for multiple-dose administration (e.g., a product that is to be administered more than once by the subject), the use of such preservatives is ubiquitous, as they prevent microbes that enter into the product after its first use from growing and infecting the patient on a later use of the product. Although providing effective biocidal properties with well-tolerated short-term use at low concentrations, preservatives can cause serious inflammatory effects on the eye with long-term use in chronic conditions, such as glaucoma, or potentially result in ocular allergies.

It has unexpectedly been found that when a liquid ophthalmic composition containing tetracaine or a pharmaceutically acceptable salt thereof is formulated for multiple-dose administration without the addition of preservatives, such as preservatives commonly used in known liquid ophthalmic compositions containing tetracaine, the composition exhibits antimicrobial activity, remains stable for an extended period of time, and is less irritating to the eye than known compositions containing tetracaine and a preservative.

Thus, provided in the present disclosure are liquid ophthalmic compositions containing tetracaine or a pharmaceutically acceptable salt thereof that are formulated for multiple-dose administration and do not contain a preservative. The preservative-free compositions of the present disclosure not only exhibit anesthetic properties, but also exhibit antimicrobial activity, remain stable for an extended period of time, and are less irritating to the eye than similarly formulated compositions that contain one or more preservatives. Also provided are methods of producing surface anesthesia of the eye in a subject in need thereof, the methods including administering to the subject the preservative-free liquid ophthalmic compositions of the present disclosure.

### Definitions

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The term “about” preceding a stated value includes the stated value and also includes ±20% of the stated value, and includes more specifically values of ±10%, ±5%, ±2%, and ±1% of the stated value.

To provide a more concise description, some of the quantitative expressions herein are recited as a range from about amount X to about amount Y. It is understood that when a range is recited, the range is not limited to the recited upper and lower bounds, but rather includes the full range from about amount X through about amount Y, or any range therein.

The compositions of the present disclosure are formulated as multiple-dose formulations. “Multiple-dose,” as used herein, means that the composition is formulated so that more than one dose can be administered from the same composition in the same container to one subject or to multiple subjects. For example, the composition can be formulated in a container that allows multiple doses to be administered from the bottle to one or to multiple subjects.

As used herein, a pharmaceutical composition of the present disclosure that contains an active agent (for example, tetracaine or a pharmaceutically acceptable salt thereof) is considered “stable” if:
(a) over the desired period of time the agents or combination of agents in the composition remain within established Quality Control-Chemistry limits for the testing parameters designed to demonstrate the stability of one or more physico-chemical aspects of the composition (including, but not limited to, appearance, activity (for example, as measured by in vitro assays), related substances/impurities, pH, and/or osmolality), and
(b) over the desired period of time the composition remains within established Quality Control-Microbiology limits for the testing parameters designed to demonstrate the stability of one or more microbiological aspects of the composition (including, but not limited to, sterility and antimicrobial effectiveness testing).

As used herein, a “pharmaceutically acceptable salt” refers to a derivative of tetracaine, wherein the tetracaine is modified by making the acid or base salt thereof of tetracaine. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound, or a tautomer thereof, formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, ethane sulfonic, fumaric, glucoheptonic, gluconic, glutamic, glycolic, glycollyarsanilic, hexylresorcinic, hydrabamic, hydrobromic, hydrochloric, hydroiodide, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicylic, stearic, subacetic, succinic, sulfamic, sulfanilic, sulfuric, tannic, tartaric, and toluene sulfonic.

“Subject,” as used herein, means a human or a non-human mammal, for example, a dog, a cat, a mouse, a rat, a cow, a sheep, a pig, a goat, a non-human primate or a bird, e.g., a chicken, as well as any other vertebrate or invertebrate. In some embodiments, the subject is a human. In some embodiments, the subject has experienced and/or exhibited at least one symptom of a disease or condition of the eye that requires anesthesia of the eye.

The term “topical” or “topically” as used here refers to application of a composition, such as a liquid ophthalmic composition of the present disclosure, to the eye, including the surface of the cornea and/or the surface of the conjunctiva, the mucosa, or the dermal area proximal to the eye. Topical administration also includes placement of the ophthalmic composition in the cul de sac of the eye. Topical application or administering can result in the delivery of an active agent to the eye.

### Liquid Ophthalmic Compositions

The present disclosure relates to preservative-free liquid ophthalmic compositions of tetracaine or a pharmaceutically acceptable salt thereof formulated for multiple doses.

Provided in the present disclosure is a preservative-free, liquid ophthalmic composition formulated for multiple-dose administration that contains tetracaine (2-(dimethylamino)ethyl 4-(butylamino)benzoate), a compound having the structure of Formula (I):

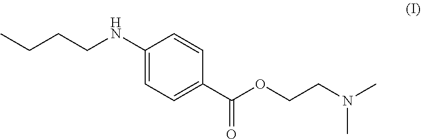 [SMILES: CCCCNc1ccc(C(=O)OCCN(C)C)cc1]

or a pharmaceutically acceptable salt thereof. In some embodiments, the composition contains tetracaine or a pharmaceutically acceptable salt thereof at a concentration of about 0.2% to about 1.0% (w/v). In some embodiments, the composition is disposed within a sterilized container suitable for a sterile ophthalmic product. In some embodiments, the composition is for ophthalmic administration.

The liquid ophthalmic compositions of the present disclosure do not contain a preservative. In some embodiments, the compositions do not contain a preservative that is selected from the group consisting of chlorobutanol, benzalkonium chloride, polyhexamethylene biguanide (PHMB), methylparaben, a stabilized peroxy complex, and any other pharmaceutically acceptable preservative for a sterile ophthalmic product. In some embodiments, the composition does not contain chlorobutanol. In some embodiments, the preservative-free liquid ophthalmic compositions of the present disclosure are stable for a period of time, such as after about 6 months, about 12 months, about 24 months, about 36 months, about 48 months, or about 60 months.

The liquid ophthalmic compositions of the present disclosure contain tetracaine or a pharmaceutically acceptable salt thereof. Non-limiting examples of pharmaceutically acceptable salts of tetracaine include, but are not limited to, sulfate, pyrosulfate, bisulfate, sulfite, bisulfite, phosphate, monohydrogen phosphate, dihydrogen phosphate, metaphosphate, pyrophosphate, hydrochloride, hydrobromide, hydroiodide, acetate, nitrate, propionate, decanoate, caprylate, acrylate, formate, isobutyrate, caprate, heptanoate, propiolate, oxalate, malonate, succinate, tocopheryl succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylenesulfonate, phenylacetate, phenylpropionate, phenylbutyrate, citrate, lactate, β-hydroxybutyrate, glycollate, tartrate, methanesulfonate, propanesulfonate, naphthalene-2-sulfonate, p-toluenesulfonate and mandelate salts, and combinations of any two or more of the foregoing. In some embodiments, the tetracaine salt is tetracaine hydrochloride.

In some embodiments, the liquid ophthalmic composition contains about 0.1% to about 1% (w/v) tetracaine or a pharmaceutically acceptable salt thereof, such as about 0.1% to about 0.9%, about 0.1% to about 0.8%, about 0.1% to about 0.7%, about 0.1% to about 0.6%, about 0.1% to about 0.5%, about 0.1% to about 0.4%, about 0.1% to about 0.3%, about 0.1% to about 0.2%, about 0.2% to about 1%, about 0.2% to about 0.9%, about 0.2% to about 0.8%, about 0.2% to about 0.7%, about 0.2% to about 0.6%, about 0.2% to about 0.5%, about 0.2% to about 0.4%, about 0.2% to about 0.3%, about 0.3% to about 1%, about 0.3% to about 0.9%, about 0.3% to about 0.8%, about 0.3% to about 0.7%, about 0.3% to about 0.6%, about 0.3% to about 0.5%, about 0.3% to about 0.4%, about 0.4% to about 1%, about 0.4% to about 0.9%, about 0.4% to about 0.8%, about 0.4% to about 0.7%, about 0.4% to about 0.6%, about 0.4% to about 0.5%, about 0.5% to about 1%, about 0.5% to about 0.9%, about 0.5% to about 0.8%, about 0.5% to about 0.7%, about 0.5% to about 0.6%, about 0.6% to about 1%, about 0.6% to about 0.9%, about 0.6% to about 0.8%, about 0.6% to about 0.7%, about 0.7% to about 1%, about 0.7% to about 0.9%, about 0.7% to about 0.8%, about 0.8% to about 1%, about 0.8% to about 0.9%, about 0.9% to about 1%, or about 0.1%, about 0.2%, about 0.3%, about 0.4%, about 0.44%, about 0.5%, about 0.6%, about 0.7%, about 0.8%, about 0.9%, or about 1% (w/v) tetracaine or a pharmaceutically acceptable salt thereof In some embodiments, the liquid ophthalmic composition contains about 0.4% to about 0.6% (w/v) tetracaine or a pharmaceutically acceptable salt thereof In some embodiments, the liquid ophthalmic composition contains about 0.5% (w/v) tetracaine or a pharmaceutically acceptable salt thereof. In some embodiments, the liquid ophthalmic composition contains about 0.44% (w/v) tetracaine or a pharmaceutically acceptable salt thereof. In some embodiments, the tetracaine is the tetracaine free base and is present in the liquid ophthalmic composition in an amount of about 0.44% (w/v). In some embodiments, the tetracaine is tetracaine hydrochloride and is present in the liquid ophthalmic composition in an amount of about 0.5% (w/v).

In some embodiments, the liquid ophthalmic composition contains a tonicity agent. Examples of suitable tonicity agents include, but are not limited to potassium chloride, sodium chloride, glycerin, lactose, mannitol, dextrose, sodium sulfate, sorbitol, saline-sodium citrate (SSC), and combinations thereof. In some embodiments, the tonicity agent is potassium chloride.

In some embodiments, the liquid ophthalmic composition contains about 0.01% to about 1% (w/v) of a tonicity agent, such as about 0.01% to about 0.9%, about 0.01% to about 0.8%, about 0.01% to about 0.7%, about 0.01% to about 0.6%, about 0.01% to about 0.5%, about 0.01% to about 0.4%, about 0.01% to about 0.3%, about 0.01% to about 0.2%, about 0.01% to about 0.1%, about 0.01% to about 0.05%, about 0.05% to about 1%, about 0.05% to about 0.9%, about 0.05% to about 0.8%, about 0.05% to about 0.7%, about 0.05% to about 0.6%, about 0.05% to about 0.5%, about 0.05% to about 0.4%, about 0.05% to about 0.3%, about 0.05% to about 0.2%, about 0.05% to about 0.1%, about 0.1% to about 1%, about 0.1% to about 0.9%, about 0.1% to about 0.8%, about 0.1% to about 0.7%, about 0.1% to about 0.6%, about 0.1% to about 0.5%, about 0.1% to about 0.4%, about 0.1% to about 0.3%, about 0.1% to about 0.2%, about 0.2% to about 1%, about 0.2% to about 0.9%, about 0.2% to about 0.8%, about 0.2% to about 0.7%, about 0.2% to about 0.6%, about 0.2% to about 0.5%, about 0.2% to about 0.4%, about 0.2% to about 0.3%, about 0.3% to about 1%, about 0.3% to about 0.9%, about 0.3% to about 0.8%, about 0.3% to about 0.7%, about 0.3% to about 0.6%, about 0.3% to about 0.5%, about 0.3% to about 0.4%, about 0.4% to about 1%, about 0.4% to about 0.9%, about 0.4% to about 0.8%, about 0.4% to about 0.7%, about 0.4% to about 0.6%, about 0.4% to about 0.5%, about 0.5% to about 1%, about 0.5% to about 0.9%, about 0.5% to about 0.8%, about 0.5% to about 0.7%, about 0.5% to about 0.6%, about 0.6% to about 1%, about 0.6% to about 0.9%, about 0.6% to about 0.8%, about 0.6% to about 0.7%, about 0.7% to about 1%, about 0.7% to about 0.9%, about 0.7% to about 0.8%, about 0.8% to about 1%, about 0.8% to about 0.9%, about 0.9% to about 1%, or about 0.01%, about 0.05%, about 0.1%, about 0.108%, about 0.11%, about 0.2%, about 0.3%, about 0.4%, about 0.44%, about 0.5%, about 0.6%, about 0.7%, about 0.8%, about 0.9%, or about 1% (w/v) tonicity agent. In some embodiments, the liquid ophthalmic composition contains about 0.05% to about 0.5% (w/v) tonicity agent. In some embodiments, the liquid ophthalmic composition contains about 0.108% (w/v) tonicity agent. In some embodiments, the tonicity agent is potassium chloride and the liquid ophthalmic composition contains about 0.05% to about 0.5(w/v) potassium chloride. In some embodiments, the tonicity agent is potassium chloride and the liquid ophthalmic composition contains about 0.108% (w/v) potassium chloride.

In some embodiments, the liquid ophthalmic composition contains a buffering agent. Buffering agents are added to keep the pH constant. Examples of suitable buffering agents include, but are not limited to boric acid, sodium borate, magnesium hydroxide, aluminum hydroxide, citric acid, sodium citrate, tartrate buffer, phosphate buffer, acetate buffer, and tris-HCl buffer (comprising tris(hydroxymethyl)aminomethane and HCl). In some embodiments, the buffering agent is boric acid.

In some embodiments, the liquid ophthalmic composition contains about 0.1% to about 2% (w/v) of a buffering agent, such as about 0.1% to about 1.9%, about 0.1% to about 1.8%, about 0.1% to about 1.7%, about 0.1% to about 1.6%, about 0.1% to about 1.5%, about 0.1% to about 1.4%, about 0.1% to about 1.3%, about 0.1% to about 1.2%, about 0.1% to about 1.1%, about 0.1% to about 1%, about 0.1% to about 0.5%, about 0.5% to about 2%, about 0.5% to about 1.9%, about 0.5% to about 1.8%, about 0.5% to about 1.7%, about 0.5% to about 1.6%, about 0.5% to about 1.5%, about 0.5% to about 1.4%, about 0.5% to about 1.3%, about 0.5% to about 1.2%, about 0.5% to about 1.1%, about 0.5% to about 1%, about 1% to about 2%, about 1% to about 1.9%, about 1% to about 1.8%, about 1% to about 1.7%, about 1% to about 1.6%, about 1% to about 1.5%, about 1% to about 1.4%, about 1% to about 1.3%, about 1% to about 1.2%, about 1% to about 1.1%, about 1.1% to about 2%, about 1.1% to about 1.9%, about 1.1% to about 1.8%, about 1.1% to about 1.7%, about 1.1% to about 1.6%, about 1.1% to about 1.5%, about 1.1% to about 1.4%, about 1.1% to about 1.3%, about 1.1% to about 1.2%, about 1.2% to about 2%, about 1.2% to about 1.9%, about 1.2% to about 1.8%, about 1.2% to about 1.7%, about 1.2% to about 1.6%, about 1.2% to about 1.5%, about 1.2% to about 1.4%, about 1.2% to about 1.3%, about 1.3% to about 2%, about 1.3% to about 1.9%, about 1.3% to about 1.8%, about 1.3% to about 1.7%, about 1.3% to about 1.6%, about 1.3% to about 1.5%, about 1.3% to about 1.4%, about 1.4% to about 2%, about 1.4% to about 1.9%, about 1.4% to about 1.8%, about 1.4% to about 1.7%, about 1.4% to about 1.6%, about 1.4% to about 1.5%, about 1.5% to about 2%, about 1.5% to about 1.9%, about 1.5% to about 1.8%, about 1.5% to about 1.7%, about 1.5% to about 1.6%, about 1.6% to about 2%, about 1.6% to about 1.9%, about 1.6% to about 1.8%, about 1.6% to about 1.7%, about 1.7% to about 2%, about 1.7% to about 1.9%, about 1.7% to about 1.8%, about 1.8% to about 2%, about 1.8% to about 1.9%, about 1.9% to about 2%, or about 0.1%, about 0.5%, about 1%, about 1.1%, about 1.2%, about 1.24%, about 1.3%, about 1.4%, about 1.5%, about 1.6%, about 1.7%, about 1.8%, about 1.9%, or about 2% (w/v) buffering agent. In some embodiments, the liquid ophthalmic composition contains about 0.1% to about 2% (w/v) buffering agent. In some embodiments, the liquid ophthalmic composition contains about 1.24% (w/v) buffering agent. In some embodiments, the buffering agent is boric acid and the liquid ophthalmic composition contains about 0.1% to about 2% (w/v) boric acid. In some embodiments, the buffering agent is boric acid and the liquid ophthalmic composition contains about 1.24% (w/v) boric acid.

In some embodiments, the liquid ophthalmic composition contains a chelating agent. A chelating agent used herein refers to a chemical compound that has the ability to react with a metal ion to form a complex through one or more bonds. The one or more bonds are typically ionic or coordination bonds. The chelating agent can be an inorganic or an organic compound. A metal ion capable of catalyzing certain chemical reactions (e.g., oxidation reactions) may lose its catalytic activity when the metal ion is bound to a chelating agent to form a complex. Therefore, a chelating agent may show preservative properties when it binds to a metal ion. Any suitable chelating agent that has preservative properties can be used, such as phosphonic acids, aminocarboxylic acids, hydroxycarboxylic acids, polyamines, aminoalcohols, and polymeric chelating agents. Specific examples of chelating agents include, but are not limited to, ethylenediaminetetraacetic acid (EDTA), edetate disodium, nitrilotriacetic acid (NTA), diethylenetriaminepentacetic acid (DTPA), N-hydroxyethylethylene diaminetriacetic acid (HEDTA), tetraborates, triethylamine diamine, and salts and derivatives thereof. In some embodiments, the chelating agent is edetate disodium.

In some embodiments, the liquid ophthalmic composition contains about 0.01% to about 0.5% (w/v) of a chelating agent, such as about 0.01% to about 0.25%, about 0.01% to about 0.1%, about 0.01% to about 0.09%, about 0.01% to about 0.08%, about 0.01% to about 0.07%, about 0.01% to about 0.06%, about 0.01% to about 0.05%, about 0.01% to about 0.04%, about 0.01% to about 0.03%, about 0.01% to about 0.02% about 0.02% to about 0.5%, about 0.02% to about 0.25%, about 0.02% to about 0.1%, about 0.02% to about 0.09%, about 0.02% to about 0.08%, about 0.02% to about 0.07%, about 0.02% to about 0.06%, about 0.02% to about 0.05%, about 0.02% to about 0.04%, about 0.02% to about 0.03%, about 0.03% to about 0.5%, about 0.03% to about 0.25%, about 0.03% to about 0.1%, about 0.03% to about 0.09%, about 0.03% to about 0.08%, about 0.03% to about 0.07%, about 0.03% to about 0.06%, about 0.03% to about 0.05%, about 0.03% to about 0.04%, about 0.04% to about 0.5%, about 0.04% to about 0.25%, about 0.04% to about 0.1%, about 0.04% to about 0.09%, about 0.04% to about 0.08%, about 0.04% to about 0.07%, about 0.04% to about 0.06%, about 0.04% to about 0.05%, about 0.05% to about 0.5%, about 0.05% to about 0.25%, about 0.05% to about 0.1%, about 0.05% to about 0.09%, about 0.05% to about 0.08%, about 0.05% to about 0.07%, about 0.05% to about 0.06%, about 0.06% to about 0.5%, about 0.06% to about 0.25%, about 0.06% to about 0.1%, about 0.06% to about 0.09%, about 0.06% to about 0.08%, about 0.06% to about 0.07%, about 0.07% to about 0.5%, about 0.07% to about 0.25%, about 0.07% to about 0.1%, about 0.07% to about 0.09%, about 0.07% to about 0.08%, about 0.08% to about 0.5%, about 0.08% to about 0.25%, about 0.08% to about 0.1%, about 0.08% to about 0.09%, about 0.09% to about 0.5%, about 0.09% to about 0.25%, about 0.09% to about 0.1%, about 0.1% to about 0.5%, about 0.1% to about 0.25%, about 0.25% to about 0.5%, or about 0.01%, about 0.02%, about 0.03%, about 0.04%, about 0.05%, about 0.06%, about 0.07%, about 0.08%, about 0.09%, about 0.1%, about 0.2%, about 0.25%, about 0.3%, about 0.4%, or about 0.5%, (w/v) chelating agent.

In some embodiments, the liquid ophthalmic composition contains about 0.02% to about 0.08% (w/v) chelating agent. In some embodiments, the liquid ophthalmic composition contains about 0.05% (w/v) chelating agent. In some embodiments, the chelating agent is edetate disodium and the liquid ophthalmic composition contains about 0.02% to about 0.08% (w/v) edetate disodium. In some embodiments, the chelating agent is edetate disodium and the liquid ophthalmic composition contains about 0.05% (w/v) edetate disodium.

In some embodiments, the liquid ophthalmic composition has a pH of about 3 to about 6.5, such as about 3 to about 6, about 3 to about 5.5, about 3 to about 5, about 3 to about 4.5, about 3 to about 4, about 3 to about 3.5, about 3.5 to about 6.5, about 3.5 to about 6, about 3.5 to about 5.5, about 3.5 to about 5, about 3.5 to about 4.5, about 3.5 to about 4, about 3.7 to about 6.5, about 3.7 to about 6, about 3.7 to about 5.5, about 3.7 to about 5, about 3.7 to about 4.5, about 3.7 to about 4, about 4 to about 6.5, about 4 to about 6, about 4 to about 5.5, about 4 to about 5, about 4 to about 4.5, about 4.5 to about 6.5, about 4.5 to about 6, about 4.5 to about 5.5, about 4.5 to about 5, about 5 to about 6.5, about 5 to about 6, about 5 to about 5.5, about 5.5 to about 6.5, about 5.5 to about 6, about 6 to about 6.5, or about 3, about 3.5, about 4, about 4.5, about 5, about 5.5, about 6, or about 6.5. In some embodiments, the liquid ophthalmic composition has a pH of about 3 to about 6.5. In some embodiments, the liquid ophthalmic composition has a pH of about 3.7 to about 6. In some embodiments, the liquid ophthalmic composition has a pH of about 3 to about 5.5. In some embodiments, the liquid ophthalmic composition has a pH of about 5.5.

In some embodiments, the liquid ophthalmic composition contains a pH adjuster. In some embodiments, the pH adjuster is an acid. In some embodiments, the pH adjuster is a base. In some embodiments, the pH adjuster is a combination of an acid and a base. Examples of suitable pH adjusters include, but are not limited to, hydrochloric acid, phosphorus acid, sulfuric acid, boric acid, citric acid, malic acid, tartaric acid, acetic acid, sodium hydroxide, potassium hydroxide, ammonium hydroxide, and tromethamine. In some embodiments, the pH adjuster is a combination of sodium hydroxide and hydrochloric acid.

In some embodiments, the liquid ophthalmic composition is formulated as a total volume of about 1 mL to about 30 mL, such as about 1 mL to about 25 mL, about 1 mL to about 20 mL, about 1 mL to about 15 mL, about 1 mL to about 10 mL, about 1 mL to about 9 mL, about 1 mL to about 8 mL, about 1 mL to about 7 mL, about 1 mL to about 6 mL, about 1 mL to about 5 mL, about 1 mL to about 4 mL, about 1 mL to about 3 mL, about 1 mL to about 2 mL, about 2 mL to about 30 mL, about 2 mL to about 25 mL, about 2 mL to about 20 mL, about 2 mL to about 15 mL, about 2 mL to about 10 mL, about 2 mL to about 9 mL, about 2 mL to about 8 mL, about 2 mL to about 7 mL, about 2 mL to about 6 mL, about 2 mL to about 5 mL, about 2 mL to about 4 mL, about 2 mL to about 3 mL, about 3 mL to about 30 mL, about 3 mL to about 25 mL, about 3 mL to about 20 mL, about 3 mL to about 15 mL, about 3 mL to about 10 mL, about 3 mL to about 9 mL, about 3 mL to about 8 mL, about 3 mL to about 7 mL, about 3 mL to about 6 mL, about 3 mL to about 5 mL, about 3 mL to about 4 mL, about 4 mL to about 30 mL, about 4 mL to about 25 mL, about 4 mL to about 20 mL, about 4 mL to about 15 mL, about 4 mL to about 10 mL, about 4 mL to about 9 mL, about 4 mL to about 8 mL, about 4 mL to about 7 mL, about 4 mL to about 6 mL, about 4 mL to about 5 mL, about 5 mL to about 30 mL, about 5 mL to about 25 mL, about 5 mL to about 20 mL, about 5 mL to about 15 mL, about 5 mL to about 10 mL, about 5 mL to about 9 mL, about 5 mL to about 8 mL, about 5 mL to about 7 mL, about 5 mL to about 6 mL, about 6 mL to about 30 mL, about 6 mL to about 25 mL, about 6 mL to about 20 mL, about 6 mL to about 15 mL, about 6 mL to about 10 mL, about 6 mL to about 9 mL, about 6 mL to about 8 mL, about 6 mL to about 7 mL, about 7 mL to about 30 mL, about 7 mL to about 25 mL, about 7 mL to about 20 mL, about 7 mL to about 15 mL, about 7 mL to about 10 mL, about 7 mL to about 9 mL, about 7 mL to about 8 mL, about 8 mL to about 30 mL, about 8 mL to about 25 mL, about 8 mL to about 20 mL, about 8 mL to about 15 mL, about 8 mL to about 10 mL, about 8 mL to about 9 mL, about 9 mL to about 30 mL, about 9 mL to about 25 mL, about 9 mL to about 20 mL, about 9 mL to about 15 mL, about 9 mL to about 10 mL, about 10 mL to about 30 mL, about 10 mL to about 25 mL, about 10 mL to about 20 mL, about 10 mL to about 15 mL, or about 1 mL, about 2 mL, about 3 mL, about 4 mL, about 5 mL, about 6 mL, about 7 mL, about 8 mL, about 9 mL, about 10 mL, about 11 mL, about 12 mL, about 13 mL, about 14 mL, about 15 mL, about 16 mL, about 17 mL, about 18 mL, about 19 mL, about 20 mL, about 21 mL, about 22 mL, about 23 mL, about 24 mL, about 25 mL, about 26 mL, about 27 mL, about 28 mL, about 29 mL, or about 30 mL. In some embodiments, the liquid ophthalmic composition is formulated as a total volume of about 1 mL to about 30 mL. In some embodiments, the liquid ophthalmic composition is formulated as a total volume of about 2 mL to about 15 mL. In some embodiments, the liquid ophthalmic composition is formulated as a total volume of about 1 mL, about 2 mL, about 4 mL, about 10 mL, about 15 mL, or about 30 mL. In some embodiments, the liquid ophthalmic composition is formulated as a total volume of about 1 mL. In some embodiments, the liquid ophthalmic composition is formulated as a total volume of about 2 mL. In some embodiments, the liquid ophthalmic composition is formulated as a total volume of about 4 mL. In some embodiments, the liquid ophthalmic composition is formulated as a total volume of about 10 mL. In some embodiments, the liquid ophthalmic composition is formulated as a total volume of about 15 mL. In some embodiments, the liquid ophthalmic composition is formulated as a total volume of about 30 mL.

The liquid ophthalmic composition of the present disclosure can be sterilized. In some embodiments, the compositions are sterilized for use in humans. The goal is to provide a safe pharmaceutical product, free of infection causing micro-organisms. The FDA has provided regulatory guidance in the publication “Guidance for Industry: Sterile Drug Products Produced by Aseptic Processing” available at: http://www.fda.gov/cder/guidance/5882fn1.htm, which is incorporated herein by reference in its entirety. As used herein, sterilization means a process used to destroy or remove microorganisms that are present in a product or packaging. Any suitable method for sterilization of objects and compositions can be used. Suitable methods for the inactivation of microorganisms include, but are not limited to, the application of extreme heat, lethal chemicals, filtration, electron beam, or gamma radiation. In some embodiments, a process for the preparation of an ophthalmic formulation comprises subjecting the formulation to a sterilization method selected from heat sterilization, chemical sterilization, radiation sterilization, electron beam sterilization, and filtration sterilization. The method used depends largely upon the nature of the device or composition to be sterilized. Methods by which the sterility of an ophthalmic composition can be assured are established in USP Chapter <1211>, which is incorporated herein by reference in its entirety.

In some embodiments, the sterilized liquid ophthalmic compositions are free of microorganisms. Acceptable sterility levels are based on applicable standards that define therapeutically acceptable compositions, including but not limited to, USP Chapter <71>, which is incorporated herein by reference in its entirety. Acceptable sterility requires no growth of any microorganisms. Acceptable sterility requires the exclusion of all microbiological agents.

In some embodiments, the liquid ophthalmic composition is disposed within a sterilized container, for example, a sterilized container suitable for multiple-dose administration of a sterile ophthalmic product. In some embodiments, the container is a multiple-dose container. The container can be any container suitable for multiple-dose administration, such as a multi-dose plastic container closure system (CCS). In some embodiments, the container is a low density polyethylene (LDPE) bottle. In other embodiments, the container is a polypropylene (PP) or high density polyethylene (HDPE) bottle. In yet other embodiments, the container is a glass bottle

For the FDA to allow a sterile drug product to be marketed in a multiple-dose container, the product must demonstrate antimicrobial effectiveness as required by the USP. See, United States Pharmacopoeia 42, National Formulary 37 (USP 42-NF 37) (2019) Chapter <51> Antimicrobial Effectiveness Testing. Antimicrobial preservatives are substances added to sterile dosage forms to protect them from microbiological growth or from microorganisms that are introduced inadvertently during or subsequent to the manufacturing process. In the case of sterile articles packaged in multiple-dose containers, antimicrobial preservatives are added to inhibit the growth of microorganisms that may be introduced from repeatedly withdrawing individual doses. Antimicrobial effectiveness, whether from the addition of an antimicrobial preservative or a result of inherent properties of the product, must be demonstrated for all multiple-dose topical dosage forms.

The requirements for antimicrobial effectiveness are met if the criteria specified in USP 42-NF 37 (2019) Chapter <51> are met. The Antimicrobial Effectiveness Test is conducted by adding specified microorganisms individually to the test product at relatively high concentrations to simulate contamination. The product is held for 28 days, during which time the added microorganisms are enumerated at defined intervals to determine any change in microbial content. The five cultures of microorganisms required by the USP to be tested include Candida albicans, Aspergillus brasiliensis, Escherichia coli, Pseudomonas aeruginosa, and Staphylococcus aureus. The acceptance criteria are specified for each of four compendia drug product categories. Sterile compositions, such as the liquid ophthalmic compositions of the present disclosure, are classified as Category 1 Products, which are defined as: “Injections, other parenterals, including emulsions, otic products, sterile nasal products, and ophthalmic products made with aqueous bases or other vehicles.” The criteria for USP <51> antimicrobial effectiveness of a Category 1 Product is as follows, where “no increase” in counts is defined as not more than 0.5 log₁₀ unit more than the value to which it is compared. See, United States Pharmacopoeia 42, National Formulary 37 (USP 42-NF 37), 2019, <51> Antimicrobial Effectiveness Testing, Table 3.

For Category 1 Products

| | |
|---|---|
| Bacteria | Not less than 1.0 log reduction from the initial |
| | calculated count at 7 days, not less than 3.0 log |
| | reduction from the initial calculated count at 14 days, |
| | and no increase from the 14 days' count at 28 days |
| Yeasts and | No increase from the initial calculated count at |
| Molds | 7, 14, and 28 days |

Preservative-free, liquid ophthalmic tetracaine compositions formulated for single-use topical administration are known. For example, Tetracaine Hydrochloride Ophthalmic Solution 0.5% STERI-UNITS® is available from Alcon (Fort Worth, Tex.). This formulation contains 0.5% tetracaine hydrochloride, sodium chloride, sodium acetate trihydrate, acetic acid, and water. However, because the composition is preservative-free, the user is instructed to administer one drop, then discard any unused portion. Liquid ophthalmic tetracaine compositions formulated for multiple-use topical administration are also known. To meet the USP requirements for antimicrobial effectiveness, these multi-dose compositions are not preservative-free. For example, Tetracaine Hydrochloride Ophthalmic Solution USP, 0.5% has been available from Altaire Pharmaceuticals (Aquebogue, N.Y.). This formulation contains 0.5% tetracaine hydrochloride and includes the preservative chlorobutanol. No multiple-dose, preservative-free, liquid ophthalmic compositions containing tetracaine are known. It has unexpectedly been discovered herein that the compositions of the present disclosure, compositions that are formulated for multiple-dose administration and are preservative-free, are able to demonstrate the antimicrobial effectiveness required by the USP and FDA and maintain effectiveness as a topical anesthetic.

USP Chapter <51> requires antimicrobial agents to be added to products that are packaged in containers that allow for the withdrawal or administration of multiple doses. However, if a multiple-dose drug product does not contain additional agents (e.g., antimicrobial agents or preservatives) and is sufficiently microbicidal to meet the requirements of the Antimicrobial Effectiveness Testing set forth in USP Chapter <51>, the drug product can be used in multiple-dose form without a preservative. Thus, in some embodiments, the preservative-free liquid ophthalmic compositions of the present disclosure meet the requirements set forth by the USP for multiple-dose compositions formulated without a preservative. In some embodiments, the preservative-free, liquid ophthalmic compositions of the present disclosure meet the requirements set forth by the USP for multiple-dose compositions formulated without a preservative at 7 days, 14 days, and 28 days.

In some embodiments, the preservative-free, liquid ophthalmic composition meets the requirements of USP Chapter <51> when tested immediately after manufacturing the composition and when tested at 3 months, 6 months, 9 months, and 12 months. In some embodiments, the preservative-free, liquid ophthalmic composition meets the requirements of USP Chapter <51> when tested at 12 month intervals for 12 months up to 60 months, for example, at 12 months, 24 months, 36 months, 48 months, or 60 months.

In some embodiments, the preservative-free, liquid ophthalmic composition exhibits antimicrobial preservative efficacy against bacterial and fungal (yeast and mold) organisms. For example, the composition can exhibit bacteriostatic, bactericidal, fungistatic, and fungicidal properties against a wide spectrum of microorganisms. In some embodiments, the liquid ophthalmic composition exhibits antimicrobial preservative efficacy against bacterial and fungal organisms including, but not limited to, Staphylococcus aureus, Pseudomonas aeruginosa, Escherichia coli, Candida albicans, and Aspergillus brasiliensis. In some embodiments, the preservative-free, liquid ophthalmic composition exhibits antimicrobial preservative efficacy against additional bacterial and fungal (yeast and mold) organisms besides those specified in USP Chapter <51>.

In some embodiments, the composition exhibits at least a 1-log reduction in the amount of organisms in about 7 days after exposure to the organisms for about 7 days to about 60 days as compared to the initial amount of organisms, such as for about 7 days to about 45 days, about 7 days to about 30 days, about 7 days to about 28 days, about 7 days to about 21 days, about 7 days to about 14 days, about 14 days to about 60 days, about 14 days to about 45 days, about 14 days to about 30 days, about 14 days to about 28 days, about 14 days to about 21 days, about 21 days to about 60 days, about 21 days to about 45 days, about 21 days to about 30 days, about 21 days to about 28 days, about 28 days to about 60 days, about 28 days to about 45 days, about 28 days to about 30 days, about 30 days to about 60 days, about 30 days to about 45 days, or about 45 days to about 60 days as compared to the initial amount of organisms. In some embodiments, the composition exhibits at least a 1-log reduction in the amount of organisms in about 7 days after exposure to the organisms for about 7 days as compared to the initial amount of organisms. In some embodiments, the composition exhibits at least a 1-log reduction in the amount of organisms in about 7 days after exposure to the organisms for about 14 days as compared to the initial amount of organisms. In some embodiments, the composition exhibits at least a 1-log reduction in the amount of organisms in about 7 days after exposure to the organisms for about 28 days as compared to the initial amount of organisms.

In some embodiments, the composition exhibits at least a 3-log reduction in the amount of organisms in about 14 days after exposure to the organisms for about 7 days to about 60 days as compared to the initial amount of organisms, such as for about 7 days to about 45 days, about 7 days to about 30 days, about 7 days to about 28 days, about 7 days to about 21 days, about 7 days to about 14 days, about 14 days to about 60 days, about 14 days to about 45 days, about 14 days to about 30 days, about 14 days to about 28 days, about 14 days to about 21 days, about 21 days to about 60 days, about 21 days to about 45 days, about 21 days to about 30 days, about 21 days to about 28 days, about 28 days to about 60 days, about 28 days to about 45 days, about 28 days to about 30 days, about 30 days to about 60 days, about 30 days to about 45 days, or about 45 days to about 60 days as compared to the initial amount of organisms. In some embodiments, the composition exhibits at least a 3-log reduction in the amount of organisms in about 14 days after exposure to the organisms for about 7 days as compared to the initial amount of organisms. In some embodiments, the composition exhibits at least a 3-log reduction in the amount of organisms in about 14 days after exposure to the organisms for about 14 days as compared to the initial amount of organisms. In some embodiments, the composition exhibits at least a 3-log reduction in the amount of organisms in about 14 days after exposure to the organisms for about 28 days as compared to the initial amount of organisms.

In some embodiments, the composition exhibits at least a 1-log reduction in the amount of organisms in about 7 days and a 3-log reduction in the amount of organisms in about 14 days after exposure to the organisms for about 7 days as compared to the initial amount of organisms. In some embodiments, the composition exhibits at least a 1-log reduction in the amount of organisms in about 7 days and a 3-log reduction in the amount of organisms in about 14 days after exposure to the organisms for about 14 days as compared to the initial amount of organisms. In some embodiments, the composition exhibits at least a 1-log reduction in the amount of organisms in about 7 days and a 3-log reduction in the amount of organisms in about 14 days after exposure to the organisms for about 28 days as compared to the initial amount of organisms. In some embodiments, the organisms are bacteria.

In some embodiments, the composition exhibits no increase in the amount of organisms from the initial calculated count after exposure to the organisms for about 7 days as compared to the initial amount of organisms, after exposure to the organisms for about 14 days as compared to the initial amount of organisms, after exposure to the organisms for about 21 days as compared to the initial amount of organisms, and after exposure to the organisms for about 28 days as compared to the initial amount of organisms. In some embodiments, the organisms are yeast. In some embodiments, the organisms are mold.

In some embodiments, the liquid ophthalmic composition has a shelf-life of (or is stable for) at least about 6 months, at least about 12 months, at least about 24 months, at least about 36 months, at least about 48 months, at least about 60 months, or more. For example, the compositions remain within established Quality Control-Chemistry limits for the testing parameters designed to demonstrate the stability of one or more physico-chemical aspects of the composition (including, but not limited to, appearance, activity (for example, as measured by in vitro assays), related substances/impurities, pH, and/or osmolality), and the compositions remain within established Quality Control-Microbiology limits for the testing parameters designed to demonstrate the stability of one or more microbiological aspects of the composition (including, but not limited to, sterility and antimicrobial effectiveness testing) over time, e.g., at least about 6 months, at least about 12 months, at least about 24 months, at least about 36 months, at least about 48 months, or at least about 60 months as compared to the original composition after manufacturing. In some embodiments, the compositions do not exhibit a significant change, as defined by the International Conference on Harmonisation of Technical Requirements for Registration of Pharmaceuticals for Human Use (ICH), in one or more of appearance, pH, percent impurities, activity (as measured by in vitro assays), or osmolarity over time, e.g., at least 12 months as compared to the original pharmaceutical composition after manufacturing. In some embodiments, the compositions conform to USP Chapter <71> sterility standards when tested initially after manufacturing (newly manufactured), or after at least 1 month, at least 2 months, at least 3 months, at least 6 months, at least 12 months, at least 18 months, at least 24 months, at least 36 months, 48 months, or at least 60 months. In some embodiments, the compositions conform to USP Chapter <51> antimicrobial effectiveness testing standards when tested initially after manufacturing (newly manufactured), or after at least 1 month, at least 2 months, at least 3 months, at least 6 months, at least 12 months, at least 18 months, at least 24 months, at least 36 months, 48 months, or at least 60 months. In some embodiments, the antimicrobial preservative efficacy of the composition remains effective throughout the entire shelf-life of the product. For example, the antimicrobial preservative efficacy of the composition remains effective for at least about 1 month, at least about 2 months, at least about 3 months, at least about 6 months, at least about 12 months, at least about 18 months, at least about 24 months, at least about 36 months, at least about 48 months, at least about 60 months, or more.

In some embodiments, stability (or shelf-life) can be determined by extrapolating stability data. In some embodiments, the stability data is obtained by performing accelerated stability tests. According to the ICH, if real time data are supported by results from accelerated studies, the shelf life may be extended beyond the end of real time studies. In accelerated stability tests, a product is stored at elevated stress conditions (such as temperature, humidity, and/or pH). Stability can be predicted or extrapolated using data obtained using accelerated stability tests. In some embodiments, the accelerated conditions include storing the composition at about 40° C.±2° C., such as about 38° C., about 39° C., about 40° C., about 41° C., or about 42° C. In some embodiments, the accelerated conditions include storing the composition at about 25% relative humidity. In some embodiments, the accelerated conditions include storing the composition at a pH of about 3.7 to about 6.0, such as about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, or about 6.0. In some embodiments, the accelerated conditions include storing the composition at a temperature of 40° C.±2° C., at about 25% relative humidity, and at a pH of about 4.5 to about 5.5. In some embodiments, no significant change is observed in the composition at accelerated storage conditions after storage for a particular amount of time. In some embodiments, data obtained from a composition that exhibits no significant change after storage at accelerated conditions for about 6 months can be extrapolated to give a shelf-life of about 24 months.

Provided in the present disclosure are liquid ophthalmic compositions that do not contain a preservative. In some embodiments, the liquid ophthalmic composition consists of about 0.2% to about 1.0% (w/v) tetracaine or a pharmaceutically acceptable salt thereof; about 0.05% to about 0.5% (w/v) of a tonicity agent; about 0.1% to about 2% (w/v) of a buffering agent; about 0.02% to about 0.08% (w/v) of a chelating agent; a pH adjuster; and water; where the composition is disposed within a container-closure system suitable for a sterile ophthalmic product and is formulated for multiple-dose administration. In some embodiments, the tetracaine is tetracaine hydrochloride. In some embodiments, the composition is formulated for ophthalmic administration. In some embodiments, the composition is formulated as a total volume of about 1 mL to about 30 mL or about 2 mL to about 15 mL. In some embodiments, the composition is formulated as a total volume of about 1 mL, about 2 mL, about 4 mL, about 10 mL, about 15 mL, or about 30 mL. In some embodiments, the composition has been sterilized.

In some embodiments, the liquid ophthalmic composition does not contain a preservative and consists of about 0.4% to about 0.6% (w/v) tetracaine hydrochloride; about 0.05% to about 0.5% (w/v) potassium chloride; about 0.1% to about 2% (w/v) boric acid; about 0.02% to about 0.08% (w/v) edetate disodium; a pH adjuster; and water; where the composition is disposed within a container-closure system suitable for a sterile ophthalmic product and is formulated for multiple-dose administration. In some embodiments, the composition is formulated for ophthalmic administration. In some embodiments, the composition is formulated as a total volume of about 1 mL to about 30 mL or about 2 mL to about 15 mL. In some embodiments, the composition is formulated as a total volume of about 1 mL, about 2 mL, about 4 mL, about 10 mL, about 15 mL, or about 30 mL. In some embodiments, the composition has been sterilized.

In some embodiments, the liquid ophthalmic composition does not contain a preservative and consists of 0.5% (w/v) tetracaine hydrochloride; 0.108% (w/v) potassium chloride; 1.24% (w/v) boric acid; 0.05% (w/v) edetate disodium; a pH adjuster; and water; where the composition is disposed within a container-closure system appropriate for a sterile ophthalmic product and is formulated for multiple-dose administration. In some embodiments, the composition is formulated for ophthalmic administration. In some embodiments, the composition is formulated as a total volume of about 1 mL to about 30 mL or about 2 mL to about 15 mL. In some embodiments, the composition is formulated as a total volume of about 1 mL, about 2 mL, about 4 mL, about 10 mL, about 15 mL, or about 30 mL. In some embodiments, the composition has been sterilized.

In some embodiments, the liquid ophthalmic composition is stored at a temperature of about 2° C. to about 25° C. after preparation of the composition, such as about 2° C. to about 20° C., about 2° C. to about 15° C., about 2° C. to about 10° C., about 2° C. to about 5° C., about 5° C. to about 25° C., about 5° C. to about 20° C., about 5° C. to about 15° C., about 5° C. to about 10° C., about 10° C. to about 25° C., about 10° C. to about 20° C., about 10° C. to about 15° C., about 15° C. to about 25° C., about 15° C. to about 20° C., about 20° C. to about 25° C., or about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., or about 25° C. In some embodiments, the liquid ophthalmic composition is stored at room temperature (RT), for example, at about 25° C. In some embodiments, the liquid ophthalmic composition is stored under refrigerated conditions, for example, the composition is stored at about 2° C. to about 8° C., or about 5° C.

### Methods for Producing Surface Anesthesia of the Eye

The present disclosure relates to methods of producing surface anesthesia of the eye. The methods include administering to a subject a liquid ophthalmic composition that contains tetracaine or a pharmaceutically acceptable salt thereof and does not contain a preservative. In some embodiments, the method includes administering to a subject a liquid ophthalmic composition of the present disclosure that contains about 0.2% to about 1.0% (w/v) tetracaine or a pharmaceutically acceptable salt thereof and does not contain a preservative. In some embodiments, the composition contains about 0.4% to about 0.6% (w/v) tetracaine or a pharmaceutically acceptable salt thereof. In some embodiments, the composition contains about 0.5% (w/v) tetracaine hydrochloride, or about 0.44% (w/v) tetracaine free base. In some embodiments, the composition has been sterilized. In some embodiments, the composition is disposed within a container-closure system appropriate for a sterile ophthalmic product and is formulated for multiple-dose administration. In some embodiments, the composition is formulated as a total volume of about 1 mL to about 30 mL or about 2 mL to about 15 mL. In some embodiments, the composition is formulated as a total volume of about 1 mL, about 2 mL, about 4 mL, about 10 mL, about 15 mL, or about 30 mL.

In some embodiments of the method of producing surface anesthesia of the eye, the liquid ophthalmic composition does not contain a preservative selected from chlorobutanol, benzalkonium chloride, PHMB, methylparaben, a stabilized peroxy complex, and any other pharmaceutically acceptable preservative for a sterile ophthalmic product.

Provided in the present disclosure is a method of producing surface anesthesia of the eye in a subject in need thereof, the method including administering to the subject a liquid ophthalmic composition that does not contain a preservative. In some embodiments of the method, the liquid ophthalmic composition consists of about 0.2% to about 1.0% (w/v) tetracaine or a pharmaceutically acceptable salt thereof; about 0.05% to about 0.5% (w/v) of a tonicity agent; about 0.1% to about 2% (w/v) of a buffering agent; about 0.02% to about 0.08% (w/v) of a chelating agent; a pH adjuster; and water; where the composition is disposed within a container-closure system suitable for a sterile ophthalmic product and is formulated for multiple-dose administration. In some embodiments of the method, the tetracaine is tetracaine hydrochloride. In some embodiments of the method, the composition is formulated for ophthalmic administration. In some embodiments, the composition is formulated as a total volume of about 1 mL to about 30 mL or about 2 mL to about 15 mL. In some embodiments of the method, the composition is formulated as a total volume of about 1 mL, about 2 mL, about 4 mL, about 10 mL, about 15 mL, or about 30 mL. In some embodiments of the method, the composition has been sterilized. In some embodiments of the method, the composition is administered to the eye as an eye drop.

In some embodiments of the method, the liquid ophthalmic composition does not contain a preservative and consists of about 0.4% to about 0.6% (w/v) tetracaine hydrochloride; about 0.05% to about 0.5% (w/v) potassium chloride; about 0.1% to about 2% (w/v) boric acid; about 0.02% to about 0.08% (w/v) edetate disodium; a pH adjuster; and water; where the composition is disposed within a container-closure system suitable for a sterile ophthalmic product and is formulated for multiple-dose administration. In some embodiments of the method, the composition is formulated for ophthalmic administration. In some embodiments, the composition is formulated as a total volume of about 1 mL to about 30 mL or about 2 mL to about 15 mL. In some embodiments of the method, the composition is formulated as a total volume of about 1 mL, about 2 mL, about 4 mL, about 10 mL, about 15 mL, or about 30 mL. In some embodiments of the method, the composition has been sterilized. In some embodiments of the method, the composition is administered to the eye as an eye drop.

In some embodiments of the method, the liquid ophthalmic composition does not contain a preservative and consists of 0.5% (w/v) tetracaine hydrochloride; 0.108% (w/v) potassium chloride; 1.24% (w/v) boric acid; 0.05% (w/v) edetate disodium; a pH adjuster; and water; where the composition is disposed within a container-closure system appropriate for a sterile ophthalmic product and is formulated for multiple-dose administration. In some embodiments of the method, the composition is formulated for ophthalmic administration. In some embodiments, the composition is formulated as a total volume of about 1 mL to about 30 mL or about 2 mL to about 15 mL. In some embodiments of the method, the composition is formulated as a total volume of about 1 mL, about 2 mL, about 4 mL, about 10 mL, about 15 mL, or about 30 mL. In some embodiments of the method, the composition has been sterilized. In some embodiments of the method, the composition is administered to the eye as an eye drop.

In some embodiments of any of the methods of the present disclosure, the liquid ophthalmic composition is administered to the eye as an eye drop. In some embodiments, a dose of one or two drops is administered. In some embodiments, a dose of one drop is administered. In some embodiments, a dose of two drops is administered. In some embodiments of the method, the dose is administered between 1 and 10 times, such as 1 to 7 times, 1 to 5 times, 1 to 3 times, 1 to 2 times, 2 to 10 times, 2 to 7 times, 2 to 5 times, 2 to 3 times, 3 to 10 times, 3 to 7 times, 3 to 5 times, 5 to 10 times, 5 to 7 times, 7 to 10 times, or 1 time, 2 times, 3 times, 4 times, 5 times, 6 times, 7 times, 8 times, 9 times, or 10 times. In some embodiments, the dose is administered 1 time. In some embodiments, the dose is administered 2 times.

In some embodiments of any of the methods of the present disclosure, the dose is administered more than once, for example, the dose is administered two or more times. In some embodiments, the dose is administered two or more times and is administered once about every 1-30 minutes, such as once about every 1-25 minutes, once about every 1-20 minutes, once about every 1-15 minutes, once about every 1-10 minutes, once about every 1-5 minutes, once about every 5-30 minutes, once about every 5-25 minutes, once about every 5-20 minutes, once about every 5-15 minutes, once about every 5-10 minutes, once about every 10-30 minutes, once about every 10-25 minutes, once about every 10-20 minutes, once about every 10-15 minutes, once about every 20-30 minutes, once about every 20-25 minutes, or once about every 25-30 minutes, such as once about every 1 minute, once about every 2 minutes, once about every 3 minutes, once about every 4 minutes, once about every 5 minutes, once about every 6 minutes, once about every 7 minutes, once about every 8 minutes, once about every 9 minutes, once about every 10 minutes, once about every 15 minutes, once about every 20 minutes, once about every 25 minutes, or once about every 30 minutes.

In some embodiments of any of the methods of the present disclosure, the liquid ophthalmic composition is administered to the subject prior to evaluation of the subject for a procedure involving the eye. In some embodiments, the procedure is a procedure in which a rapid and short acting topical ophthalmic anesthetic is indicated. In some embodiments of the method, the liquid ophthalmic composition is administered to the subject prior to the subject undergoing a procedure involving the eye. In some embodiments, the procedure is a procedure in which a rapid and short acting topical ophthalmic anesthetic is indicated. In some embodiments, one dose of one or two drops is administered.

The procedure can be any procedure involving the eye where a topical anesthetic can be used. Examples of procedures include, but are not limited to, tonometry, gonioscopy, removal of corneal foreign bodies, conjunctival scraping for diagnostic purposes, suture removal from the cornea or conjunctiva, cataract extraction, and other short corneal and conjunctival procedures. In some embodiments, the procedure is tonometry or other short corneal and conjunctival procedures. In some embodiments, the procedure is removal of corneal foreign bodies or suture removal from the cornea or conjunctiva. In some embodiments, the procedure is cataract extraction.

Thus, in some embodiments of any of the methods of the present disclosure, the procedure is tonometry or other short corneal and conjunctival procedures and one dose of one or two drops is administered to the subject prior to evaluation of the subject. In other embodiments of the method, the procedure is removal of corneal foreign bodies or suture removal from the cornea or conjunctiva and the dose is administered to the subject once about every 5-10 minutes for one to three times. In yet other embodiments, the procedure is cataract extraction and the dose is administered to the subject once about every 5-10 minutes for three to five times.

### EXAMPLES

### Example 1—Preservative Effectiveness Testing and Re-Challenge Test of Preservative-Free 0.5% Tetracaine Hydrochloride Ophthalmic Solution

The antimicrobial properties of a preservative-free 0.5% tetracaine hydrochloride ophthalmic solution were tested according to the Preservative Effectiveness Test (PET) from USP Chapter <51> and re-challenged during the test. The preservative efficacy of the tetracaine solution was tested against bacterial and fungal organisms that included bacteria (Staphylococcus aureus, Pseudomonas aeruginosa, and Escherichia coli), yeast (Candida albicans), and mold (Aspergillus brasiliensis).

Briefly, the USP Chapter <51> Antimicrobial Effectiveness Test was conducted by adding the specified microorganisms individually to the test product (tetracaine formulation) at relatively high concentrations to simulate contamination. The product was held for 28 days, during which time the added microorganisms were enumerated at defined intervals to determine any change in microbial content.

A panel of five challenge organisms were used, including Candida albicans (yeast), Aspergillus brasiliensis (mold), Escherichia coli (Gram-negative enterobacillus), Pseudomonas aeruginosa (Gram-negative bacillus), and Staphylococcus aureus (Gram-positive coccus). Fresh cultures of each organism were harvested in sterile saline and standardized to about 10⁸ colony forming units per mL (cfu/mL). The microbial enumeration test was performed to determine the number of viable cells in each cell suspension. Bacteria were grown at 30° C. to 35° C. on Soybean-Casein Digest Agar, while yeast and mold were grown at 20° C. to 25° C. on Sabouraud Dextrose Agar. Table 1 shows the culture conditions for the preparation of standardized cell suspensions and microbial recovery study.

TABLE 1
Incubation temperature and time for preparation of standardized
cell suspensions and microbial recovery study

| | | Temper- | Time | |
|---|---|---|---|---|
| | | ature | (cell | Time |
| Organism | Culture Medium | (° C.) | suspension) | (recovery) |
| E. coli | Soybean-Casein | 30-35 | 18-24 hours | 3-5 days |
| | Digest (broth, agar) | | | |
| P. aeruginosa | Soybean-Casein | 30-35 | 18-24 hours | 3-5 days |
| | Digest (broth, agar) | | | |
| S. aureus | Soybean-Casein | 30-35 | 18-24 hours | 3-5 days |
| | Digest (broth, agar) | | | |
| C. albicans | Sabouraud Dextrose | 20-25 | 44-52 hours | 3-5 days |
| | (broth, agar) | | | |
| A. brasiliensis | Sabouraud Dextrose | 20-25 | 6-10 days | 3-7 days |
| | (broth, agar) | | | |

The standardized cell suspensions were added to the test product in five separate containers, one container for each challenge organism. The concentration of challenge organisms in product Category 1 was between 10⁵ and 10⁶ cfu/mL. The inoculum volume did not exceed 1% of the total volume of the product tested. Inoculated samples were incubated at 20° C. to 25° C. (for yeast and mold) for 28 days, and at 30° C. to 35° C. (for bacteria) for 28 days. The microbial enumeration test was performed at days 7, 14, and 28 by the validated method.

Four separate tetracaine formulations (Formulations A-D) tested were prepared as shown in Table 2. None of the formulations contained preservative.

TABLE 2
Composition of Formulations A-D

| Ingredients | mg/mL | Percentage (%) |
|---|---|---|
| Purified Water, USP | Qs. to 1.000 mL | Qs. 100% |
| Boric Acid, NF | 12.40 | 1.24% |
| Potassium Chloride, USP | 1.08 | 0.108% |
| Edetate Disodium, USP | 0.50 | 0.05% |
| Tetracaine Hydrochloride, USP | 5.00 | 0.5% |
| Sodium Hydroxide, 10%, NF | Qs. pH | Qs. pH |
| and/or | | |
| Hydrochloride Acid, 10%, NF | | |

The USP Preservative Efficacy Test was performed on Formulations A-D. Re-challenge of the five microorganisms was performed at 14 days on Formulation D as follows. Fourteen days after the initial inoculation, 20 mL aliquots of each microorganism of the inoculated product were removed. Each product aliquot was re-inoculated with the appropriate amount of corresponding microbial suspension to yield a cell concentration of 1×10⁴ and 1×10⁵ cfu/mL. The results are shown in Tables 3-6.

TABLE 3
Log reductions achieved by Formulation A

| | E. | Pseudomonas | Staphylococcus | Candida | Aspergillus |
|---|---|---|---|---|---|
| Interval | Coli | Aeruginosa | Aureus | Albicans | brasiliensis |
| Log | 5.52 | 4.49 | 4.61 | 5.57 | 3.73 |
| reduction- | | | | | |
| 7 days | | | | | |
| Log | 5.52 | 5.49 | 5.61 | 5.57 | 5.20 |
| reduction- | | | | | |
| 14 days | | | | | |
| Log | | | | | |
| reduction- | 5.52 | 5.49 | 5.61 | 5.57 | 5.20 |
| 28 days | | | | | |

TABLE 4
Log reductions achieved by Formulation B

| | E. | Pseudomonas | Staphylococcus | Candida | Aspergillus |
|---|---|---|---|---|---|
| Interval | Coli | Aeruginosa | Aureus | Albicans | brasiliensis |
| Log | 5.52 | 5.49 | 5.61 | 5.57 | 3.60 |
| reduction- | | | | | |
| 7 days | | | | | |
| Log | 5.52 | 5.49 | 5.61 | 5.57 | 5.20 |
| reduction- | | | | | |
| 14 days | | | | | |
| Log | 5.52 | 5.49 | 5.61 | 5.57 | 5.20 |
| reduction- | | | | | |
| 28 days | | | | | |

TABLE 5
Log reductions achieved by Formulation C

| | E. | Pseudomonas | Staphylococcus | Candida | Aspergillus |
|---|---|---|---|---|---|
| Interval | Coli | Aeruginosa | Aureus | Albicans | brasiliensis |
| Log | 5.52 | 5.49 | 5.61 | 5.57 | 3.43 |
| reduction- | | | | | |
| 7 days | | | | | |
| Log | 5.52 | 5.49 | 5.61 | 5.57 | 5.20 |
| reduction- | | | | | |
| 14 days | | | | | |
| Log | 5.52 | 5.49 | 5.61 | 5.57 | 5.20 |
| reduction- | | | | | |
| 28 days | | | | | |

TABLE 6
Log reductions achieved by Formulation D

| | E. | Pseudomonas | Staphylococcus | Candida | Aspergillus |
|---|---|---|---|---|---|
| Interval | Coli | Aeruginosa | Aureus | Albicans | brasiliensis |
| Log | 5.41 | 5.76 | 5.76 | 4.51 | 5.18 |
| reduction- | | | | | |
| 7 days | | | | | |
| Log | 5.41 | 5.76 | 5.76 | 5.51 | 5.18 |
| reduction- | | | | | |
| 14 days* | | | | | |
| Log | 5.41 | 5.76 | 5.76 | 5.51 | 5.18 |
| reduction- | | | | | |
| 28 days* | | | | | |

*Re-challenge was conducted at 14 days after the plate count

The following acceptance criteria provided by the USP for Preservative Efficacy Challenges as applicable to sterile products is as follows:
(a) Bacteria—not less than 1.0 log reduction from the initial calculated count at 7 days, not less than 3.0 log reduction from the initial count at 14 days, and no increase from the 14 days count at 28 days;
(b) Yeast and Molds—no increase from the initial calculated count at 7, 14, and 28 days.

The above USP acceptance criteria were also applied to the 14-day re-challenge. The results must conform to the above acceptance criteria at the corresponding days in order to pass the test.

As shown in Tables 3-6, all of the preservative-free tetracaine formulations that were tested met the criteria established in USP Chapter <51>, demonstrating the anti-microbial efficacy of the preservative-free formulation against all five challenged microorganisms.

### Example 2—Time Kill Kinetic Study of Microorganisms in Preservative-Free 0.5% Tetracaine Hydrochloride Ophthalmic Solution

A study was performed to test the time kill rate of five representative American Type Culture Collection (ATCC) microorganisms as indicated in the USP <51> Antimicrobial Effectiveness Testing using a preservative-free 0.5% tetracaine hydrochloride ophthalmic solution (see Table 2). The microorganisms tested included Escherichia coli (ATCC 8739), Pseudomonas aeruginosa (ATCC 9027), Staphylococcus aureus (ATCC 6538), Candida albicans (ATCC 10231), and Aspergillus brasiliensis (ATCC 16404). The microbicidal properties of the tetracaine solution were also determined.

The time kill study was performed as follows. After five minutes exposure, plate counts were immediately performed for each of the five samples (5 minute exposure time samples). A serial dilution was performed in which dilution factors of 10¹, 10², 10³, and 10⁴ of the five minute exposure time samples were prepared using 0.9% saline solution with 1 mL sterile pipettes and milk dilution bottles. The dilutions were plated into Trypticase Soy Agar (bacteria) or Sabouraud Dextrose Agar (SDA) (yeast and mold) using sterile Petri plates. The plates were incubated according to the following schedule:

| | | Incubation | Incubation |
|---|---|---|---|
| Organism | Medium | Temperature | Time |
| Escherichia coli | TSA | 30° C.-35° C. | 24-48 hours |
| (ATCC 8739) | | | |
| Pseudomonas aeruginosa | TSA | 30° C.-35° C. | 24-48 hours |
| (ATCC 9027) | | | |
| Staphylococcus aureus | TSA | 30° C.-35° C. | 24-48 hours |
| (ATCC 6358) | | | |
| Candida albicans | SDA | 20° C.-25° C. | 3-5 days |
| (ATCC 10231) | | | |
| Aspergillus brasiliensis | SDA | 20° C.-25° C. | 3-7 days |
| (ATCC 16404) | | | |

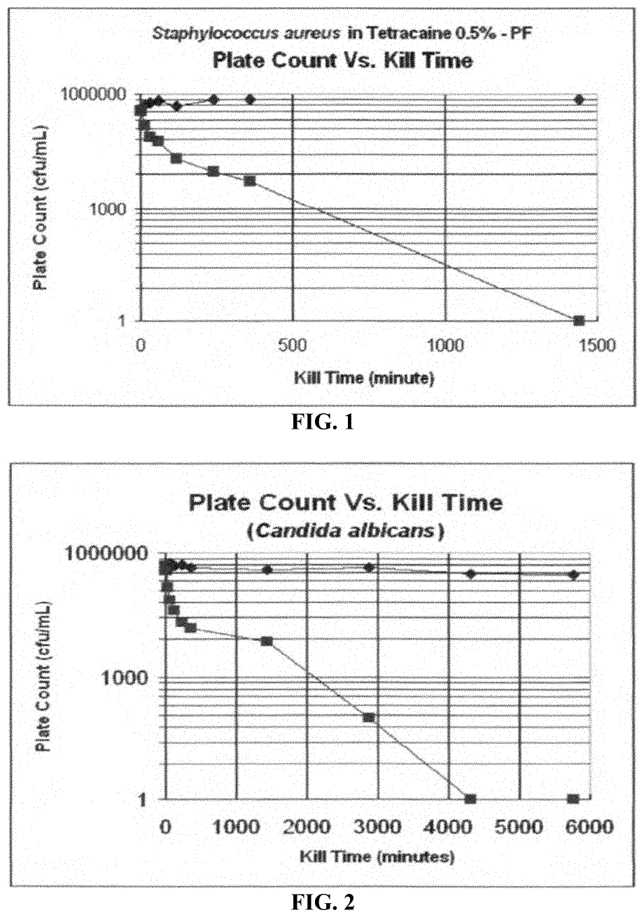

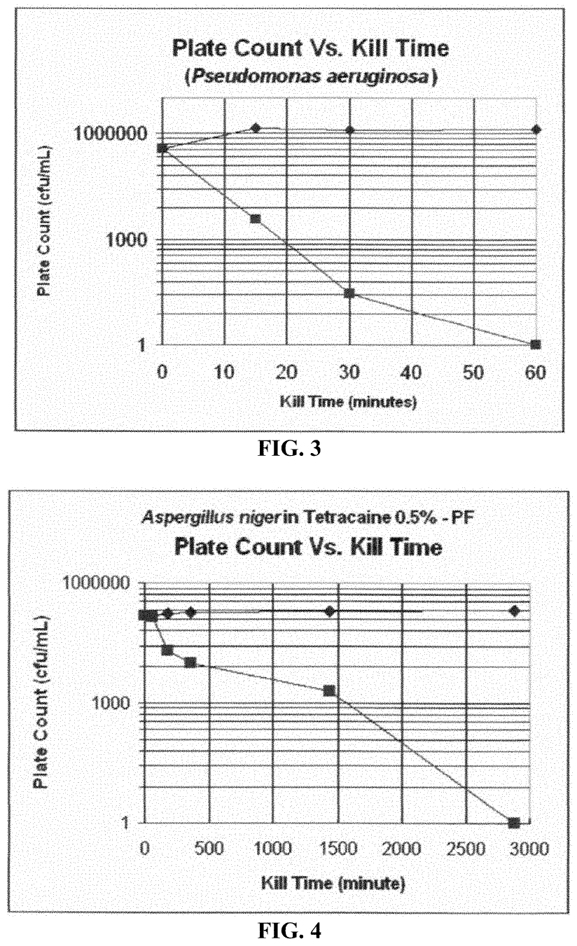

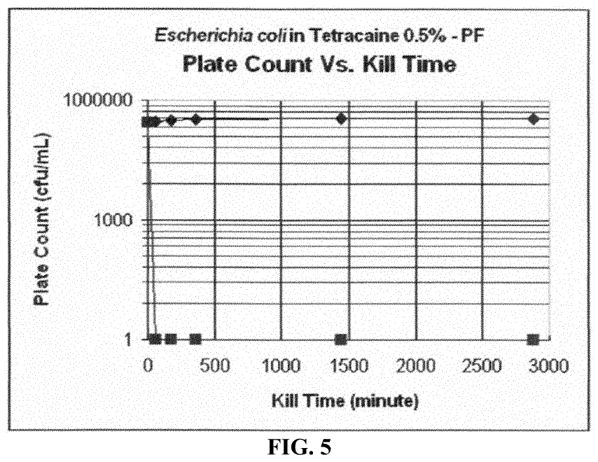

After ten minutes, samples were collected and plate counts were immediately performed for each of the samples (10 minute exposure time samples). A serial dilution was again performed in which dilution factors of 10¹, 10², 10³, and 10⁴ of the ten minute exposure time samples were prepared using 0.9% saline solution with 1 mL sterile pipettes and milk dilution bottles and the dilutions were plated on sterile Petri plates. Samples were obtained at 15 minutes, 20 minutes, 45 minutes, and 60 minutes, then plated and incubated. The same was done with a control sample (0.9% saline). Counting frequencies of all of the plates were obtained using the plate count technique. The time kill plate counts of each of the five microorganisms are shown in Tables 7-11 and plots of the plate counts as a function of time are shown in FIGS. 1-5.

TABLE 7
Time kill plate counts of S. aureus (ATCC 6538)

| | Control | | |
|---|---|---|---|
| Kill Time (min) | (saline 0.9%) | Sample | Log reduction |
| 0 | 350000 | 350000 | 0.00 |
| 5 | 430000 | 352000 | 0.00 |
| 15 | 530000 | 155000 | 0.35 |
| 30 | 610000 | 75000 | 0.67 |
| 60 | 670000 | 56000 | 0.80 |
| 120 | 500000 | 20000 | 1.24 |
| 240 | 740000 | 9300 | 1.58 |
| 360 | 740000 | 5100 | 1.84 |
| 1440 | 740000 | 0 | >5.54 |

TABLE 8
Time kill plate counts of C. Albicans (ATCC 10231)

| | Control | | |
|---|---|---|---|
| Kill Time (min) | (saline 0.9%) | Sample | Log reduction |
| 0 | 365000 | 36500 | 0.00 |
| 5 | 390000 | 500000 | −0.14 |
| 15 | 420000 | 380000 | −0.02 |
| 30 | 398000 | 148000 | 0.39 |
| 60 | 570000 | 72000 | 0.70 |
| 120 | 490000 | 39000 | 0.97 |
| 240 | 520000 | 20200 | 1.26 |
| 360 | 440000 | 15100 | 1.38 |
| 1440 | 390000 | 6900 | 1.72 |
| 2880 | 440000 | 105 | 3.54 |
| 4320 | 310000 | 0 | >5.56 |
| 5760 | 300000 | 0 | >5.56 |

TABLE 9
Time kill plate counts of P. aeruginosa (ATCC 9027)

| | Control | | |
|---|---|---|---|
| Kill Time (min) | (saline 0.9%) | Sample | Log reduction |
| 0 | 365000 | 365000 | 0.00 |
| 15 | 1400000 | 3670 | 2.00 |
| 30 | 1260000 | 30 | 4.09 |
| 60 | 1270000 | 0 | >5.56 |

TABLE 10
Time kill plate counts of A. brasiliensis (ATCC 16404)

| | Control | | |
|---|---|---|---|
| Kill Time (min) | (saline 0.9%) | Sample | Log reduction |
| 0 | 150000 | 150000 | 0.00 |
| 60 | 150000 | 140000 | 0.03 |
| 180 | 170000 | 20000 | 0.88 |
| 360 | 180000 | 10000 | 1.18 |
| 1440 | 200000 | 2000 | 1.88 |
| 2880 | 210000 | 0 | >5.18 |

TABLE 11
Time kill plate counts of E. coli (ATCC 8739)

| | Control | | |
|---|---|---|---|
| Kill Time (min) | (saline 0.9%) | Sample | Log reduction |
| 0 | 270000 | 270000 | 0.00 |
| 60 | 300000 | 0 | >5.43 |
| 180 | 320000 | 0 | >5.43 |
| 360 | 330000 | 0 | >5.43 |
| 1440 | 350000 | 0 | >5.43 |
| 2880 | 360000 | 0 | >5.43 |

Table 12 summarizes the time kill data for each of the five microorganisms tested. As Table 12 shows, the preservative-free tetracaine solution achieved a 3-log reduction in the population of the tested microorganisms in as little as 22 minutes (P. aeruginosa) and a greater than 5-log reduction in 60 minutes (P. aeruginosa and E. coli).

TABLE 12
Time kill data

| Microorganism | 3 log kill time | | >5 log kill time | |
|---|---|---|---|---|
| Escherichia coli | 25 | minutes | 60 | minutes |
| (ATCC 8739) | | | | |
| Pseudomonas aeruginosa | 22 | minutes | 60 | minutes |
| (ATCC 9027) | | | | |
| Staphylococcus aureus | 12 | hours | 24 | hours |
| (ATCC 6358) | | | | |
| Candida albicans | 40 | hours | 72 | hours |
| (ATCC 10231) | | | | |
| Aspergillus brasiliensis | 27 | hours | 48 | hours |
| (ATCC 16404) | | | | |

### Example 3—Stability Study of Preservative-Free 0.5% Tetracaine Hydrochloride Ophthalmic Solution

Three separate formulations (Formulations E-G) of a preservative-free 0.5% tetracaine hydrochloride ophthalmic solution were prepared according to Table 13. Each of Formulations E-G were prepared as a 4 mL sample in 5 cc white low density polyethylene (LDPE) Boston Round (B/R) bottles fitted with a 15 mm natural LDPE controlled dropper tip and a 15/415 white polypropylene (PP) cap and a 15 mL sample in 15 cc white LDPE B/R bottles fitted with a 15 mm natural LDPE controlled dropper tip and a 15/415 white PP cap. The samples were tested at several time points after storage under various stability conditions (refrigerated, controlled room temperature, intermediate storage, and accelerated storage). The samples were also tested by HPLC at each time point to determine whether degradation of the tetracaine had occurred.

TABLE 13
Composition of preservative-free Formulations E-G

| Ingredients | mg/mL | Percentage (%) |
|---|---|---|
| Purified Water, USP | Qs. to 1.000 mL | Qs. 100% |
| Boric Acid, NF | 12.40 | 1.24% |
| Potassium Chloride, USP | 1.08 | 0.108% |
| Edetate Disodium, USP | 0.50 | 0.05% |
| Tetracaine Hydrochloride, USP | 5.00 | 0.5% |
| Sodium Hydroxide, 10%, NF | Qs. pH | Qs. pH |
| and/or | | |
| Hydrochloride Acid, 10%, NF | | |

Compositions formulated for multiple-dose administration that do not contain a preservative must demonstrate sufficient antimicrobial effectiveness at several time points after preparation and storage of the composition under various conditions. The compositions must conform to the specifications set forth for sterility in USP Chapter <71> and antimicrobial effectiveness in USP Chapter <51>. The acceptance criteria set forth in USP Chapter <51> that demonstrates antimicrobial effectiveness for an ophthalmic composition requires that the composition demonstrate not less than (NLT) 1.0 log reduction from the initial calculated count at 7 days, NLT 3.0 log reduction from the initial calculated count at 14 days and no increase from the 14 days' count at 28 days for bacteria; and no increase from the initial calculated count at 7, 14 and 28 days for yeasts and molds.

### Refrigerated Storage

4 mL and 15 mL samples of each of Formulations E-G were prepared and stored at 5° C.±3° C. Aliquots were removed from each sample at the initial time-point (0 months), and after 3 months, 6 months, 9 months, and 12 months of storage. The samples were tested for sterility at 0 and 12 months and antimicrobial effectiveness at 0, 3, and 12 months (see Example 1 for testing procedure). The results are shown in Tables 14-19.

TABLE 14
Stability test results of a 4 mL sample of Formulation E stored under
refrigerated conditions (5° C. ± 3° C.)

| Test | Specification | 0 month | 3 month | 6 month | 9 month | 12 month |
|---|---|---|---|---|---|---|
| Tetracaine HCl | 90.0%-110.0% | 100.9% | 101.7% | 101.8% | 102.0% | 101.7% |
| assay | | | | | | |
| USP <71> Sterility | Meets current USP | Conforms | N/R | N/R | N/R | Conforms |
| | Requirements | | | | | |
| USP <51> | Meets current USP | Conforms | Conforms | N/R | N/R | Conforms |
| Antimicrobial | Requirements | | | | | |
| Effectiveness | | | | | | |
| Testing | | | | | | |

TABLE 15
Stability test results of a 4 mL sample of Formulation F stored under
refrigerated conditions (5° C. ± 3° C.)

| Test | Specification | 0 month | 3 month | 6 month | 9 month | 12 month |
|---|---|---|---|---|---|---|
| Tetracaine HCl | 90.0%-110.0% | 100.1% | 101.5% | 101.8% | 102.4% | 98.5% |
| assay | | | | | | |
| USP <71> Sterility | Meets current USP | Conforms | N/R | N/R | N/R | Conforms |
| | Requirements | | | | | |
| USP <51> | Meets current USP | Conforms | Conforms | N/R | N/R | Conforms |
| Antimicrobial | Requirements | | | | | |
| Effectiveness | | | | | | |
| Testing | | | | | | |

TABLE 16
Stability test results of a 4 mL sample of Formulation G stored under
refrigerated conditions (5° C. ± 3° C.)

| Test | Specification | 0 month | 3 month | 6 month | 9 month | 12 month |
|---|---|---|---|---|---|---|
| Tetracaine HCl | 90.0%-110.0% | 100.2% | 101.3% | 101.8% | 103.2% | 97.6% |
| assay | | | | | | |
| USP <71> Sterility | Meets current USP | Conforms | N/R | N/R | N/R | Conforms |
| | Requirements | | | | | |
| USP <51> | Meets current USP | Conforms | Conforms | N/R | N/R | Conforms |
| Antimicrobial | Requirements | | | | | |
| Effectiveness | | | | | | |
| Testing | | | | | | |

TABLE 17
Stability test results of a 15 mL sample of Formulation E stored under
refrigerated conditions (5° C. ± 3° C.)

| Test | Specification | 0 month | 3 month | 6 month | 9 month | 12 month |
|---|---|---|---|---|---|---|
| Tetracaine HCl | 90.0%-110.0% | 100.9% | 103.0% | 99.6% | 102.2% | 99.4% |
| assay | | | | | | |
| USP <71> Sterility | Meets current USP | Conforms | N/R | N/R | N/R | Conforms |
| | Requirements | | | | | |
| USP <51> | Meets current USP | Conforms | Conforms | N/R | N/R | Conforms |
| Antimicrobial | Requirements | | | | | |
| Effectiveness | | | | | | |
| Testing | | | | | | |

TABLE 18
Stability test results of a 15 mL sample of Formulation F stored under
refrigerated conditions (5° C. ± 3° C.)

| Test | Specification | 0 month | 3 month | 6 month | 9 month | 12 month |
|---|---|---|---|---|---|---|
| Tetracaine HCl | 90.0%-110.0% | 100.7% | 103.0% | 102.3% | 102.2% | 101.2% |
| assay | | | | | | |
| USP <71> Sterility | Meets current USP | Conforms | N/R | N/R | N/R | Conforms |
| | Requirements | | | | | |
| USP <51> | Meets current USP | Conforms | Conforms | N/R | N/R | Conforms |
| Antimicrobial | Requirements | | | | | |
| Effectiveness | | | | | | |
| Testing | | | | | | |

TABLE 19
Stability test results of a 15 mL sample of Formulation G stored under
refrigerated conditions (5° C. ± 3° C.)

| Test | Specification | 0 month | 3 month | 6 month | 9 month | 12 month |
|---|---|---|---|---|---|---|
| Tetracaine HCl | 90.0%-110.0% | 101.3% | 102.% | 102.7% | 102.9% | 102.4% |
| assay | | | | | | |
| USP <71> Sterility | Meets current USP | Conforms | N/R | N/R | N/R | Conforms |
| | Requirements | | | | | |
| USP <51> | Meets current USP | Conforms | Conforms | N/R | N/R | Conforms |
| Antimicrobial | Requirements | | | | | |
| Effectiveness | | | | | | |
| Testing | | | | | | |

### Controlled Room Temperature (RT) Storage

4 mL and 15 mL samples of each of Formulations E-G were prepared and stored at 25° C.±2° C. and 40% relative humidity (RH)±5%. Aliquots were removed from each sample at the initial time-point (0 months), and after 3 months, 6 months, 9 months, and 12 months of storage. The samples were tested for sterility at 0 and 12 months and antimicrobial effectiveness at 0, 3, and 12 months (see Example 1 for testing procedure). The results are shown in Tables 20-25.

TABLE 20
Stability test results of a 4 mL sample of Formulation E stored under controlled
RT conditions (25° C. ± 2° C./40% RH ± 5%)

| Test | Specification | 0 month | 3 month | 6 month | 9 month | 12 month |
|---|---|---|---|---|---|---|
| Tetracaine HCl | 90.0%-110.0% | 100.9% | 100.3% | 99.8% | 99.3% | 100.9% |
| assay | | | | | | |
| USP <71> Sterility | Meets current USP | Conforms | N/R | N/R | N/R | Conforms |
| | Requirements | | | | | |
| USP <51> | Meets current USP | Conforms | Conforms | N/R | N/R | Conforms |
| Antimicrobial | Requirements | | | | | |
| Effectiveness | | | | | | |
| Testing | | | | | | |

TABLE 21
Stability test results of a 4 mL sample of Formulation F stored under controlled
RT conditions (25° C. ± 2° C./40% RH ± 5%)

| Test | Specification | 0 month | 3 month | 6 month | 9 month | 12 month |
|---|---|---|---|---|---|---|
| Tetracaine HCl | 90.0%-110.0% | 100.1% | 100.4% | 99.7% | 99.7% | 97.8% |
| assay | | | | | | |
| USP <71> Sterility | Meets current USP | Conforms | N/R | N/R | N/R | Conforms |
| | Requirements | | | | | |
| USP <51> | Meets current USP | Conforms | Conforms | N/R | N/R | Conforms |
| Antimicrobial | Requirements | | | | | |
| Effectiveness | | | | | | |
| Testing | | | | | | |

TABLE 22
Stability test results of a 4 mL sample of Formulation G stored under controlled
RT conditions (25° C. ± 2° C./40% RH ± 5%)

| Test | Specification | 0 month | 3 month | 6 month | 9 month | 12 month |
|---|---|---|---|---|---|---|
| Tetracaine HCl | 90.0%-110.0% | 100.2% | 100.3% | 98.8% | 100.6% | 97.5% |
| assay | | | | | | |
| USP <71> Sterility | Meets current USP | Conforms | N/R | N/R | N/R | Conforms |
| | Requirements | | | | | |
| USP <51> | Meets current USP | Conforms | Conforms | N/R | N/R | Conforms |
| Antimicrobial | Requirements | | | | | |
| Effectiveness | | | | | | |
| Testing | | | | | | |

TABLE 23
Stability test results of a 15 mL sample of Formulation E stored under controlled
RT conditions (25° C. ± 2° C./40% RH ± 5%)

| Test | Specification | 0 month | 3 month | 6 month | 9 month | 12 month |
|---|---|---|---|---|---|---|
| Tetracaine HCl | 90.0%-110.0% | 100.9% | 101.0% | 99.6% | 99.2% | 99.9% |
| assay | | | | | | |
| USP <71> Sterility | Meets current USP | Conforms | N/R | N/R | N/R | Conforms |
| | Requirements | | | | | |
| USP <51> | Meets current USP | Conforms | Conforms | N/R | N/R | Conforms |
| Antimicrobial | Requirements | | | | | |
| Effectiveness | | | | | | |
| Testing | | | | | | |

TABLE 24
Stability test results of a 15 mL sample of Formulation F stored under controlled
RT conditions (25° C. ± 2° C./40% RH ± 5%)

| Test | Specification | 0 month | 3 month | 6 month | 9 month | 12 month |
|---|---|---|---|---|---|---|
| Tetracaine HCl | 90.0%-110.0% | 100.7% | 100.9% | 100.9% | 99.5% | 100.6% |
| assay | | | | | | |
| USP <71> Sterility | Meets current USP | Conforms | N/R | N/R | N/R | Conforms |
| | Requirements | | | | | |
| USP <51> | Meets current USP | Conforms | Conforms | N/R | N/R | Conforms |
| Antimicrobial | Requirements | | | | | |
| Effectiveness | | | | | | |
| Testing | | | | | | |

TABLE 25
Stability test results of a 15 mL sample of Formulation G stored under controlled
RT conditions (25° C. ± 2° C./40% RH ± 5%)

| Test | Specification | 0 month | 3 month | 6 month | 9 month | 12 month |
|---|---|---|---|---|---|---|
| Tetracaine HCl | 90.0%-110.0% | 101.3% | 100.7% | 102.8% | 100.1% | 102.7% |
| assay | | | | | | |
| USP <71> Sterility | Meets current USP | Conforms | N/R | N/R | N/R | Conforms |
| | Requirements | | | | | |
| USP <51> | Meets current USP | Conforms | Conforms | N/R | N/R | Conforms |
| Antimicrobial | Requirements | | | | | |
| Effectiveness | | | | | | |
| Testing | | | | | | |

### Intermediate Storage Conditions

4 mL and 15 mL samples of each of Formulations E-G were prepared and stored at 30° C.±2° C. and 65% RH±5%. Aliquots were removed from each sample at the initial time-point (0 months), and after 1 month, 2 months, 3 months, 9 months, and 12 months of storage. The samples were tested for sterility at 0 months and antimicrobial effectiveness at 0, 3, and 12 months (see

Example 1 for testing procedure). The results are shown in Tables 26-31.

TABLE 26
Stability test results of a 4 mL sample of Formulation E stored under
intermediate storage conditions (30° C. ± 2° C./65% RH ± 5%)

| Test | Specification | 0 month | 1 month | 2 month | 3 month | 9 month | 12 month |
|---|---|---|---|---|---|---|---|
| Tetracaine HCl | 90.0%-110.0% | 100.9% | 101.9% | 100.5% | 98.8% | 96.9% | 101.5% |
| assay | | | | | | | |
| USP <71> Sterility | Meets current USP | Conforms | N/R | N/R | N/R | N/R | N/R |
| | Requirements | | | | | | |
| USP <51> | Meets current USP | Conforms | N/R | N/R | Conforms | N/R | Conforms |
| Antimicrobial | Requirements | | | | | | |
| Effectiveness | | | | | | | |
| Testing | | | | | | | |

TABLE 27
Stability test results of a 4 mL sample of Formulation F stored under
intermediate storage conditions (30° C. ± 2° C./65% RH ± 5%)

| Test | Specification | 0 month | 1 month | 2 month | 3 month | 9 month | 12 month |
|---|---|---|---|---|---|---|---|
| Tetracaine HCl | 90.0%-110.0% | 100.1% | 101.8% | 99.4% | 98.7% | 97.2% | 97.9% |
| assay | | | | | | | |
| USP <71> Sterility | Meets current USP | Conforms | N/R | N/R | N/R | N/R | N/R |
| | Requirements | | | | | | |
| USP <51> | Meets current USP | Conforms | N/R | N/R | Conforms | N/R | Conforms |
| Antimicrobial | Requirements | | | | | | |
| Effectiveness | | | | | | | |
| Testing | | | | | | | |

TABLE 28
Stability test results of a 4 mL sample of Formulation G stored under
intermediate storage conditions (30° C. ± 2° C./65% RH ± 5%)

| Test | Specification | 0 month | 1 month | 2 month | 3 month | 9 month | 12 month |
|---|---|---|---|---|---|---|---|
| Tetracaine HCl | 90.0%-110.0% | 100.1% | 101.9% | 101.0% | 98.7% | 97.6% | 97.3% |
| assay | | | | | | | |
| USP <71> Sterility | Meets current USP | Conforms | N/R | N/R | N/R | N/R | N/R |
| | Requirements | | | | | | |
| USP <51> | Meets current USP | Conforms | N/R | N/R | Conforms | N/R | Conforms |
| Antimicrobial | Requirements | | | | | | |
| Effectiveness | | | | | | | |
| Testing | | | | | | | |

TABLE 29
Stability test results of a 15 mL sample of Formulation E stored
under intermediate storage conditions (30° C. ± 2° C./65% RH ± 5%)

| Test | Specification | 0 month | 1 month | 2 month | 3 month | 9 month | 12 month |
|---|---|---|---|---|---|---|---|
| Tetracaine HCl | 90.0%-110.0% | 100.9% | 102.0% | 101.3% | 99.4% | 96.8% | 99.4% |
| assay | | | | | | | |
| USP <71> Sterility | Meets current USP | Conforms | N/R | N/R | N/R | N/R | N/R |
| | Requirements | | | | | | |
| USP <51> | Meets current USP | Conforms | N/R | N/R | Conforms | N/R | Conforms |
| Antimicrobial | Requirements | | | | | | |
| Effectiveness | | | | | | | |
| Testing | | | | | | | |

TABLE 30
Stability test results of a 15 mL sample of Formulation F stored
under intermediate storage conditions (30° C. ± 2° C./65% RH ± 5%)

| Test | Specification | 0 month | 1 month | 2 month | 3 month | 9 month | 12 month |
|---|---|---|---|---|---|---|---|
| Tetracaine HCl | 90.0%-110.0% | 100.7% | 101.8% | 100.3% | 99.3% | 97.1% | 101.0% |
| assay | | | | | | | |
| USP <71> Sterility | Meets current USP | Conforms | N/R | N/R | N/R | N/R | N/R |
| | Requirements | | | | | | |
| USP <51> | Meets current USP | Conforms | N/R | N/R | Conforms | N/R | Conforms |
| Antimicrobial | Requirements | | | | | | |
| Effectiveness | | | | | | | |
| Testing | | | | | | | |

TABLE 31
Stability test results of a 15 mL sample of Formulation G stored
under intermediate storage conditions (30° C. ± 2° C./65% RH ± 5%)

| Test | Specification | 0 month | 1 month | 2 month | 3 month | 9 month | 12 month |
|---|---|---|---|---|---|---|---|
| Tetracaine HCl | 90.0%-110.0% | 101.3% | 101.8% | 101.3% | 99.4% | 97.8% | 103.2% |
| assay | | | | | | | |
| USP <71> Sterility | Meets current USP | Conforms | N/R | N/R | N/R | N/R | N/R |
| | Requirements | | | | | | |
| USP <51> | Meets current USP | Conforms | N/R | N/R | Conforms | N/R | Conforms |
| Antimicrobial | Requirements | | | | | | |
| Effectiveness | | | | | | | |
| Testing | | | | | | | |

### Accelerated Storage Conditions

4 mL and 15 mL samples of each of Formulations E-G were prepared and stored at 40° C.±2° C. and NMT 25% Relative Humidity (RH). Aliquots were removed from each sample at the initial time-point (0 months), and after 1 months, 2 months, 3 months, and 6 months of storage. The samples were tested for sterility at 0 months and antimicrobial effectiveness at 0, 3, and 6 months (see Example 1 for testing procedure). The results are shown in Tables 32-37.

TABLE 32
Stability test results of a 4 mL sample of Formulation E stored under accelerated
storage conditions (40° C. ± 2° C. and NMT 25 RH %)

| Test | Specification | 0 month | 1 month | 2 month | 3 month | 6 month |
|---|---|---|---|---|---|---|
| Tetracaine HCl | 90.0%-110.0% | 100.9% | 102.1% | 99.1% | 96.0% | 99.8% |
| assay | | | | | | |
| USP <71> Sterility | Meets current USP | Conforms | N/R | N/R | N/R | N/R |
| | Requirements | | | | | |
| USP <51> | Meets current USP | Conforms | N/R | N/R | Conforms | Conforms |
| Antimicrobial | Requirements | | | | | |
| Effectiveness | | | | | | |
| Testing | | | | | | |

TABLE 33
Stability test results of a 4 mL sample of Formulation F stored under accelerated
storage conditions (40° C. ± 2° C. and NMT 25% RH)

| Test | Specification | 0 month | 1 month | 2 month | 3 month | 6 month |
|---|---|---|---|---|---|---|
| Tetracaine HCl | 90.0%-110.0% | 100.1% | 101.9% | 98.4% | 96.0% | 99.8% |
| assay | | | | | | |
| USP <71> Sterility | Meets current USP | Conforms | N/R | N/R | N/R | N/R |
| | Requirements | | | | | |
| USP <51> | Meets current USP | Conforms | N/R | N/R | Conforms | Conforms |
| Antimicrobial | Requirements | | | | | |
| Effectiveness | | | | | | |
| Testing | | | | | | |

TABLE 34
Stability test results of a 4 mL sample of Formulation G stored under accelerated
storage conditions (40° C. ± 2° C. and NMT 25% RH)

| Test | Specification | 0 month 1 month | | 2 month | 3 month | 6 month |
|---|---|---|---|---|---|---|
| Tetracaine HCl | 90.0%-110.0% | 100.2% | 101.9% | 100.4% | 95.6% | 96.7% |
| assay | | | | | | |
| USP <71> Sterility | Meets current USP | Conforms | N/R | N/R | N/R | N/R |
| | Requirements | | | | | |
| USP <51> | Meets current USP | Conforms | N/R | N/R | Conforms | Conforms |
| Antimicrobial | Requirements | | | | | |
| Effectiveness | | | | | | |
| Testing | | | | | | |

TABLE 35
Stability test results of a 15 mL sample of Formulation E stored under accelerated
storage conditions (40° C. ± 2° C. and NMT 25% RH)

| Test | Specification | 0 month | 1 month | 2 month | 3 month | 6 month |
|---|---|---|---|---|---|---|
| Tetracaine HCl | 90.0%-110.0% | 100.9% | 102.0% | 99.7% | 100.8% | 101.9% |
| assay | | | | | | |
| USP <71> Sterility | Meets current USP | Conforms | N/R | N/R | N/R | N/R |
| | Requirements | | | | | |
| USP <51> | Meets current USP | Conforms | N/R | N/R | Conforms | Conforms |
| Antimicrobial | Requirements | | | | | |
| Effectiveness | | | | | | |
| Testing | | | | | | |

TABLE 36
Stability test results of a 15 mL sample of Formulation F stored under accelerated
storage conditions (40° C. ± 2° C. and NMT 25% RH)

| Test | Specification | 0 month | 1 month | 2 month | 3 month | 6 month |
|---|---|---|---|---|---|---|
| Tetracaine HCl | 90.0%-110.0% | 100.7% | 102.0% | 98.3% | 100.7% | 100.9% |
| assay | | | | | | |
| USP <71> Sterility | Meets current USP | Conforms | N/R | N/R | N/R | N/R |
| | Requirements | | | | | |
| USP <51> | Meets current USP | Conforms | N/R | N/R | Conforms | Conforms |
| Antimicrobial | Requirements | | | | | |
| Effectiveness | | | | | | |
| Testing | | | | | | |

TABLE 37
Stability test results of a 15 mL sample of Formulation G stored under accelerated
storage conditions (40° C. ± 2° C. and NMT 25% RH)

| Test | Specification | 0 month | 1 month | 2 month | 3 month | 6 month |
|---|---|---|---|---|---|---|
| Tetracaine HCl | 90.0%-110.0% | 101.3% | 101.9% | 99.6% | 98.4% | 99.8% |
| assay | | | | | | |
| USP <71> Sterility | Meets current USP | Conforms | N/R | N/R | N/R | N/R |
| | Requirements | | | | | |
| USP <51> | Meets current USP | Conforms | N/R | N/R | Conforms | Conforms |
| Antimicrobial | Requirements | | | | | |
| Effectiveness | | | | | | |
| Testing | | | | | | |

### Results

The results in Tables 14-37 showed that all samples tested under all stability conditions conformed to the USP standards at the required time points. All samples conformed to the USP Chapter <71> requirements indicating sterility and the USP Chapter <51> requirements indicating antimicrobial effectiveness at all required time points. These results indicated that tetracaine alone was effective as a preservative throughout the shelf-life of the composition, assuring the composition maintained sterility throughout its shelf-life.

### OTHER EMBODIMENTS

It is to be understood that the foregoing description is intended to illustrate and not limit the scope of the disclosure, which is defined by the scope of the appended claims. Other aspects, advantages, and modifications are within the scope of the following claims.

What is claimed is:

1. A liquid ophthalmic composition comprising tetracaine or a pharmaceutically acceptable salt thereof at a concentration of about 0.2% to about 1.0% (w/v) disposed within a sterilized container suitable for a sterile ophthalmic product;
wherein:
the composition is formulated for multiple-dose administration; and
the composition does not contain a preservative.

2. The composition of claim 1, wherein the composition does not contain a preservative selected from the group consisting of chlorobutanol, benzalkonium chloride, PHMB, methylparaben, a stabilized peroxy complex, and any other pharmaceutically acceptable preservative for a sterile ophthalmic product.

3. The composition of claim 1, wherein the composition is for ophthalmic administration.

4. The composition of claim 1, wherein the composition comprises about 0.4% to about 0.6% (w/v) tetracaine or a pharmaceutically acceptable salt thereof.

5. The composition of claim 1, wherein the composition comprises about 0.5% (w/v) tetracaine or a pharmaceutically acceptable salt thereof.

6. The composition of claim 1, wherein the tetracaine is tetracaine hydrochloride.

7. The composition of claim 1, wherein the composition comprises a tonicity agent at a concentration of about 0.05% to about 0.5% (w/v).

8. The composition of claim 7, wherein the tonicity agent is at a concentration of about 0.108% (w/v).

9. The composition of claim 8, wherein the tonicity agent is potassium chloride.

10. The composition of claim 1, wherein the composition comprises a buffering agent at a concentration of about 0.1% to about 2% (w/v).

11. The composition of claim 10, wherein the buffering agent is at a concentration of about 1.24% (w/v).

12. The composition of claim 11, wherein the buffering agent is boric acid.

13. The composition of claim 1, wherein the composition comprises a chelating agent at a concentration of about 0.02% to about 0.08% (w/v).

14. The composition of claim 13, wherein the chelating agent is at a concentration of about 0.05% (w/v).

15. The composition of claim 14, wherein the chelating agent is edetate disodium.

16. The composition of claim 1, wherein the pH is about 3 to about 6.5.

17. The composition of claim 16, wherein the pH is about 3.7 to about 6.

18. The composition of claim 16, wherein the pH is about 3 to about 5.5.

19. The composition of claim 16, wherein the pH is about 5.5.

20. The composition of claim 1, formulated as a total volume of about 1 mL to about 30 mL.

21. The composition of claim 20, formulated as a total volume of about 2 mL to about 15 mL.

22. The composition of claim 20, formulated as a total volume of about 1 mL, about 2 mL, about 4 mL, about 10 mL, about 15 mL or about 30 mL.

23. The composition of claim 1, wherein the composition has been sterilized.

24. The composition of claim 1, wherein the composition exhibits antimicrobial preservative efficacy against bacterial and fungal organisms selected from among Staphylococcus aureus, Pseudomonas aeruginosa, Escherichia coli, Candida albicans, and Aspergillus brasiliensis.

25. The composition of claim 24, wherein the composition exhibits not less than about a 1.0 log reduction from the initial calculated count at 7 days, not less than about a 3.0 log reduction from the initial calculated count at 14 days, and no increase from the 14 days' count at 28 days for bacteria; and no increase from the initial calculated count at 7 days, 14 days and 28 days for yeast and mold.

26. The composition of claim 25, wherein the antimicrobial preservative efficacy of the composition remains effective throughout the entire shelf-life of the product.

27. The composition of claim 26, wherein the shelf-life is at least about 6 months, at least about 12 months, at least about 24 months, at least about 36 months, at least about 48 months, or at least about 60 months.

28. A liquid ophthalmic composition consisting of:
about 0.2% to about 1.0% (w/v) tetracaine or a pharmaceutically acceptable salt thereof;
about 0.05% to about 0.5% (w/v) of a tonicity agent;
about 0.1% to about 2% (w/v) of a buffering agent;
about 0.02% to about 0.08% (w/v) of a chelating agent;
pH adjuster; and
water;
wherein the composition is disposed within a container-closure system suitable for a sterile ophthalmic product and is formulated for multiple-dose administration.

29. The liquid ophthalmic composition of claim 28, wherein the tetracaine is tetracaine hydrochloride.

30. A liquid ophthalmic composition consisting of:
about 0.4% to about 0.6% (w/v) tetracaine hydrochloride;
about 0.05% to about 0.5% (w/v) potassium chloride;
about 0.1% to about 2% (w/v) boric acid;
about 0.02% to about 0.08% (w/v) edetate disodium;
pH adjuster; and
water;
wherein the composition is disposed within a container-closure system suitable for a sterile ophthalmic product and is formulated for multiple-dose administration.

31. A liquid ophthalmic composition consisting of:
0.5% (w/v) tetracaine hydrochloride;
0.108% (w/v) potassium chloride;
1.24% (w/v) boric acid;
0.05% (w/v) edetate disodium;
pH adjuster; and
water;
wherein the composition is disposed within a container-closure system appropriate for a sterile ophthalmic product and is formulated for multiple-dose administration.

32. The composition of claim 28, wherein the composition is for ophthalmic administration.

33. The composition of claim 28, formulated as a total volume of about 1 mL to about 30 mL.

34. The composition of claim 33, formulated as a total volume of about 2 mL to about 15 mL.

35. The composition of claim 33, formulated as a total volume of about 1 mL, about 2 mL, about 4 mL, about 10 mL, about 15 mL or about 30 mL.

36. The composition of claim 28, wherein the composition has been sterilized.

37. A method of producing surface anesthesia of the eye in a subject in need thereof, comprising administering a liquid ophthalmic composition comprising tetracaine or a pharmaceutically acceptable salt thereof at a concentration of about 0.2% to about 1.0% (w/v), wherein:
the composition is disposed within a container-closure system appropriate for a sterile ophthalmic product and is formulated for multiple-dose administration; and
the composition does not contain a preservative.

38. The method of claim 37, wherein the composition does not contain the preservative chlorobutanol, benzalkonium chloride, PHMB, methylparaben, a stabilized peroxy complex, or any other pharmaceutically acceptable preservative for a sterile ophthalmic product.

39. The method of claim 37, wherein the composition comprises about 0.4% to about 0.6% (w/v) tetracaine or a pharmaceutically acceptable salt thereof

40. The method of claim 37, wherein the composition comprises about 0.5% (w/v) tetracaine hydrochloride.

41. The method of claim 37, wherein the composition is formulated as a total volume of about 1 mL to about 30 mL.

42. The method of claim 41, wherein the composition is formulated as a total volume of about 2 mL to about 15 mL.

43. The method of claim 41, wherein the composition is formulated as a total volume of about 1 mL, about 2 mL, about 4 mL, about 10 mL, about 15 mL, or about 30 mL.

44. The method of claim 37, wherein the composition has been sterilized.

45. The method of claim 37, wherein the composition is administered to the eye as an eye drop.

46. The method of claim 45, wherein a dose of one or two drops is administered.

47. The method of claim 46, wherein the dose is administered 1 to 3 times, 1 to 7 times, 1 to 5 times, 1 to 3 times, 1 to 2 times, 2 to 5 times, 2 to 3 times, 3 to 10 times, 3 to 7 times, or 3 to 5 times.

48. The method of claim 47, wherein the dose is administered one time, two times, three times, four times, five times, six times, seven times, eight times, nine times, or ten times.

49. The method of claim 48, wherein the dose is administered two or more times and is administered once about every 1-30 minutes, about every 1-15 minutes, about every 1-10 minutes, about every 1-5 minutes, about every 5-30 minutes, about every 5-15 minutes, about every 5-10 minutes, or about every 10-15 minutes.

50. The method of claim 49, wherein the dose is administered once about every 1 minute, 2 minutes, 3 minutes, 4 minutes, 5 minutes, 6 minutes, 7 minutes, 8 minutes, 9 minutes, 10 minutes, 15 minutes, 20 minutes, 25 minutes, or 30 minutes.

51. The method of claim 37, wherein the composition is administered to the subject prior to evaluation of the subject for a procedure in which a rapid and short acting topical ophthalmic anesthetic is indicated.

52. The method of claim 51, wherein one dose of one or two drops is administered.

53. The method of claim 37, wherein the composition is administered to the subject prior to the subject undergoing a procedure in which a rapid and short acting topical ophthalmic anesthetic is indicated.

54. The method of claim 53, wherein the procedure is selected from the group consisting of tonometry, gonioscopy, removal of corneal foreign bodies, conjunctival scraping for diagnostic purposes, suture removal from the cornea or conjunctiva, cataract extraction, and other short corneal and conjunctival procedures.

55. The method of claim 54, wherein the procedure is tonometry or other short corneal and conjunctival procedures and one dose of one or two drops is administered to the subject prior to evaluation of the subject.

56. The method of claim 54, wherein the procedure is removal of corneal foreign bodies or suture removal from the cornea or conjunctiva and the dose is administered to the subject once about every 5-10 minutes for one to three times.

57. The method of claim 54, wherein the procedure is cataract extraction and the dose is administered to the subject once about every 5-10 minutes for three to five times.

58. A method of producing surface anesthesia of the eye in a subject in need thereof, the method comprising administering a liquid ophthalmic composition consisting of:
about 0.2% to about 1.0% (w/v) tetracaine or a pharmaceutically acceptable salt thereof;
about 0.05% to about 0.5% (w/v) of a tonicity agent;
about 0.1% to about 2% (w/v) of a buffering agent;
about 0.02% to about 0.08% (w/v) of a chelating agent;
pH adjuster; and
water;
wherein the composition is disposed within a container-closure system appropriate for a sterile ophthalmic product and is formulated for multiple-dose administration.

59. The method of claim 58, wherein the tetracaine is tetracaine hydrochloride.

60. The method of claim 58, wherein the composition consists of:
about 0.4% to about 0.6% (w/v) tetracaine hydrochloride;
about 0.05% to about 0.5% (w/v) potassium chloride;
about 0.1% to about 2% (w/v) boric acid;
about 0.02% to about 0.08% (w/v) edetate disodium;
pH adjuster; and
water.

61. The method of claim 58, wherein the composition consists of:
0.5% (w/v) tetracaine hydrochloride;
0.108% (w/v) potassium chloride;
1.24% (w/v) boric acid;
0.05% (w/v) edetate disodium;
pH adjuster; and
water.

62. The method of claim 58, formulated as a total volume of about 1 mL to about 30 mL.

63. The method of claim 62, formulated as a total volume of about 2 mL to about 15 mL.

64. The method of claim 62, formulated as a total volume of about 1 mL, about 2 mL, about 4 mL, about 10 mL, about 15 mL, or about 30 mL.

65. The method of claim 58, wherein the composition has been sterilized.

66. The method of claim 58, wherein the composition is administered to the eye as an eye drop.

67. The method of claim 66, wherein a dose of one or two drops is administered.

68. The method of claim 67, wherein the dose is administered 1 to 3 times, 1 to 7 times, 1 to 5 times, 1 to 3 times, 1 to 2 times, 2 to 5 times, 2 to 3 times, 3 to 10 times, 3 to 7 times, or 3 to 5 times.

69. The method of claim 68, wherein the dose is administered one time, two times, three times, four times, five times, six times, seven times, eight times, nine times, or ten times.

70. The method of claim 69, wherein the dose is administered two or more times and is administered once about every 1-30 minutes, about every 1-15 minutes, about every 1-10 minutes, about every 1-5 minutes, about every 5-30 minutes, about every 5-15 minutes, about every 5-10 minutes, or about every 10-15 minutes.

71. The method of claim 70, wherein the dose is administered once about every 1 minute, 2 minutes, 3 minutes, 4 minutes, 5 minutes, 6 minutes, 7 minutes, 8 minutes, 9 minutes, 10 minutes, 15 minutes, 20 minutes, 25 minutes, or 30 minutes.

72. The method of claim 58, wherein the composition is administered to the subject prior to evaluation of the subject for a procedure in which a rapid and short acting topical ophthalmic anesthetic is indicated.

73. The method of claim 72, wherein one dose of one or two drops is administered.

74. The method of claim 58, wherein the composition is administered to the subject prior to the subject undergoing a procedure in which a rapid and short acting topical ophthalmic anesthetic is indicated.

75. The method of claim 74, wherein the procedure is selected from the group consisting of tonometry, gonioscopy, removal of corneal foreign bodies, conjunctival scraping for diagnostic purposes, suture removal from the cornea or conjunctiva, and other short corneal and conjunctival procedures.

76. The method of claim 75, wherein the procedure is tonometry or other short corneal and conjunctival procedures and one dose of one or two drops is administered to the subject prior to evaluation of the subject.

77. The method of claim 75, wherein the procedure is removal of corneal foreign bodies or suture removal from the cornea or conjunctiva and the dose is administered to the subject once about every 5-10 minutes for one to three times.

78. The method of claim 75, wherein the procedure is cataract extraction and the dose is administered to the subject once about every 5-10 minutes for three to five times.
